(19) United States
(12) Patent Application Publication
(10) Pub. No.: US 2012/0270947 A1
(43) Pub. Date: Oct. 25, 2012

(54) Short Telomere Length on Chromosome 9P is Strongly Associated with Breast Cancer Risk

(75) Inventor: Yun-Ling Zheng, North Potomac, MD (US)

(73) Assignee: Georgetown University, Washington, DC (US)

(21) Appl. No.: 13/274,920

(22) Filed: Oct. 17, 2011

Related U.S. Application Data
(63) Continuation-in-part of application No. PCT/US2010/039013, filed on Jun. 17, 2010.
(60) Provisional application No. 61/218,049, filed on Jun. 17, 2009.

(51) Int. Cl. C12Q 1/68; A61K 31/138; G01N 21/64

(52) U.S. Cl. 514/648; 435/6.14; 435/6.11

(57) ABSTRACT

Disclosed are compositions and methods related to assessing the risk of cancer, such as breast cancer, through analyzing the length of telomeres, such as chromosome 9p telomere, such as the short arm of the 9p telomere. If the 9p arm is shorter than normal, the risk of cancer is increased.

### CROSS-REFERENCE TO RELATED APPLICATIONS

This application is a continuation-in-part of International Application No. PCT/US2010/039013, filed Jun. 17, 2010, which claims benefit to U.S. Provisional Application No. 61/218,049, filed on Jun. 17, 2009. International Application No. PCT/US2010/039013, filed Jun. 17, 2010, and Provisional Application No. 61/218,049, filed on Jun. 17, 2009, are hereby incorporated herein in their entirety.

### STATEMENT REGARDING FEDERALLY SPONSORED RESEARCH OR DEVELOPMENT

This invention was made with government support under Grants P30 CA51008 awarded by the National Institutes of Health. The government has certain rights in the invention.

### BACKGROUND

Breast cancer is the most common malignancy in women (Parkin et al. Breast J 12 Suppl 1:S70-S80, 2006). In the United States, breast cancer incidence rates have been rising slowly for the past two decades, and breast cancer is the second leading cause of cancer-related death in women (Stewart et al. MMWR 53:1-108, 2004; Smigal et al. CA Cancer J Clin 56:168-183, 2006). However, there is currently no accurate method to predict who is most likely to develop the disease for individuals in general population. Of the nearly 241,000 women diagnosed each year, about 80%-90% are sporadic cases who had no family history of breast cancer and no other identifiable strong risk factors other than age and reproductive or hormonal risk factors (Lancet 358:1389-1399, 2001). In order to prevent breast cancer, there is a need to develop tools to identify women at an elevated risk, allowing women and their physicians to take a more proactive approach to reduce breast cancer burden.

Disclosed herein, a short telomere on chromosome 9p is strongly associated with breast cancer risk. Chromosome 9p telomere length, as disclosed herein, can be incorporated into the current prediction models to significantly enhance breast cancer risk prediction. Better risk assessment will improve the efficiency of both population-based preventive programs, such as screening mammography, as well as individual-based preventive strategies such as chemoprevention by targeting women who are at the greatest risk for breast cancer.

### BRIEF SUMMARY

In accordance with the purpose of this invention, as embodied and broadly described herein, this invention relates to methods for assessing cancer risk based on length of chromosome telomeres.

Additional advantages of the disclosed methods will be set forth in part in the description which follows, and in part will be understood from the description, or may be learned by practice of the disclosed methods. The advantages of the disclosed methods will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF DRAWINGS

FIG. 1 illustrates the distribution of relative telomere length (RTL) between breast cancer patients (case) and healthy women controls (control). (A) Short version of chromosome 9p, (B) long version of chromosome 9p. (C) short version of chromosome 9q, and (C) long version of chromosome 9q.

FIG. 2 represents Fluorescent in situ Hybridization of total chromosomes. A Cy-3-telomere probe (red, but shown here as small light dots) was used to stain all telomeres. A FITC-chromosome 9 Sat-III probe (green, but shown here as larger light spots) was used to stain chromosome 9.

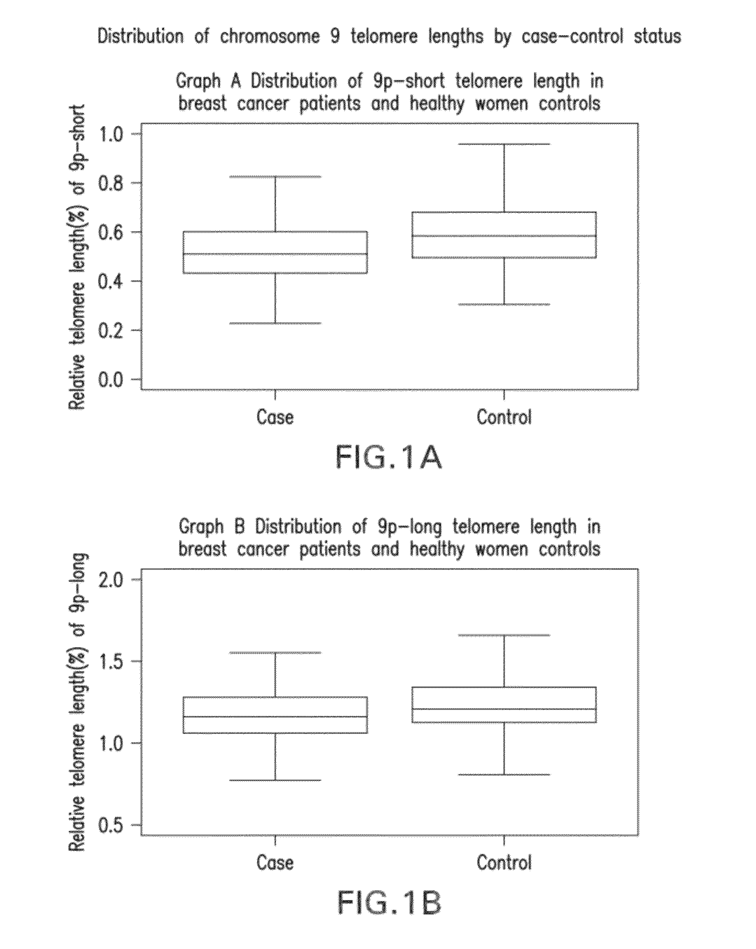

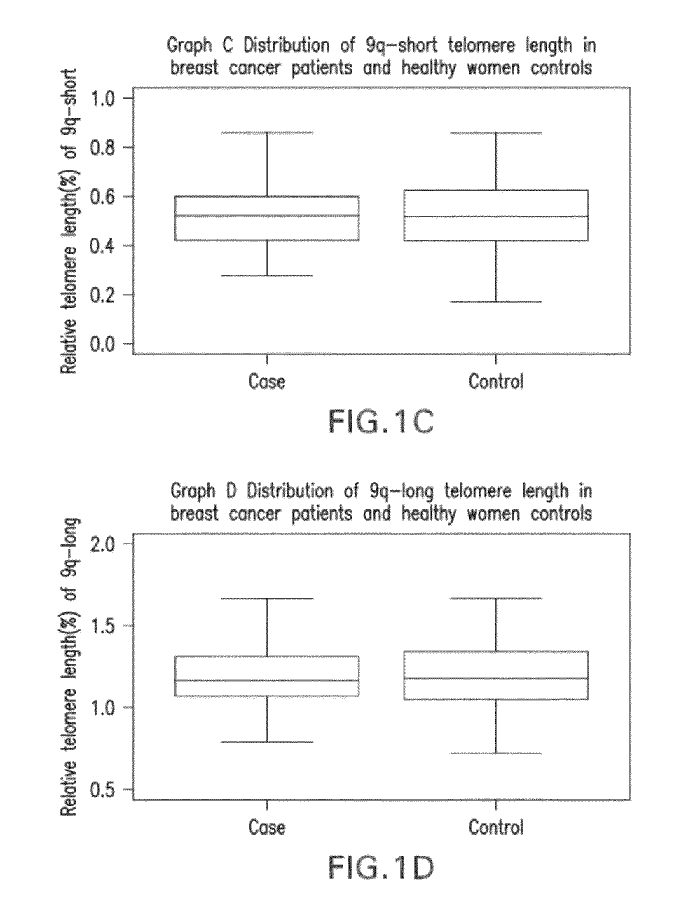

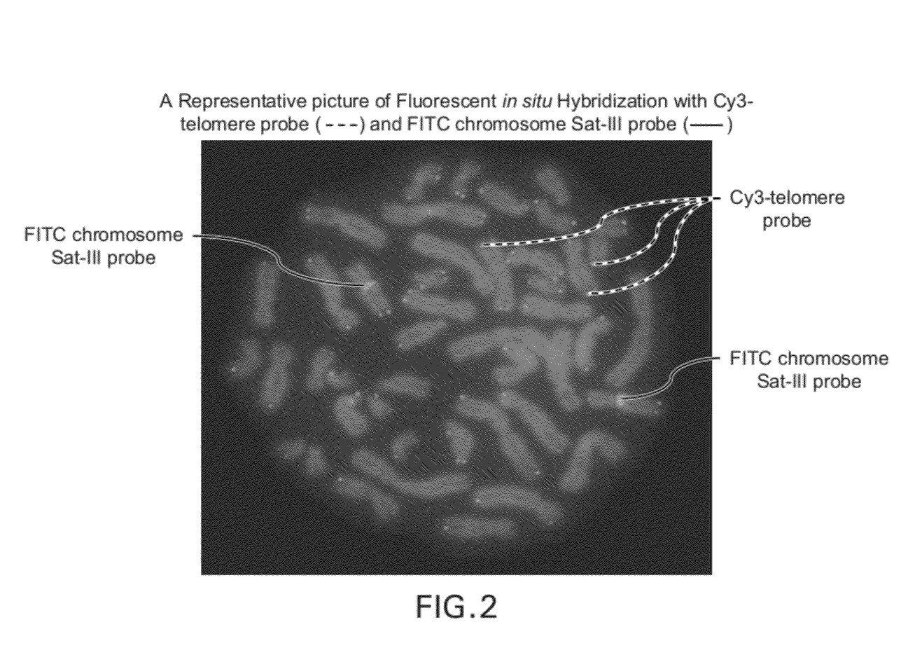

### DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described.

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

Those skilled in the art will recognize, or be able to ascertain using no more than routine experimentation, many equivalents to the specific embodiments of the method and compositions described herein. Such equivalents are intended to be encompassed by the following claims.

A. Definitions

1. Subject

As used throughout, by a “subject” is meant an individual. Thus, the “subject” can include, for example, domesticated animals, such as cats, dogs, etc., livestock (e.g., cattle, horses, pigs, sheep, goats, etc.), laboratory animals (e.g., mouse, rabbit, rat, guinea pig, etc.) mammals, non-human mammals, primates, non-human primates, rodents, birds, reptiles, amphibians, fish, and any other animal. The subject can be a mammal such as a primate or a human. The subject can also be a non-human.

2. Fluorescent

The term “fluorescent” or like terms as used herein can be defined as a molecule having luminescence that is caused by the absorption of radiation at one wavelength followed by nearly immediate reradiation usually at a different wavelength and that ceases almost at once when the incident radiation stops, as understood in the art.

3. Cancer

As used herein, the terms “cancer” and “cancerous” refer to or describe the physiological condition in mammals in which a population of cells are characterized by unregulated cell growth. Examples of cancer include, but are not limited to, carcinoma, lymphoma, blastoma, sarcoma, and leukemia. More particular examples of such cancers include squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, hepatic carcinoma and various types of head and neck cancer.

4. Treatment

By “treatment” and “treating” is meant the medical management of a subject with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. It is understood that treatment, while intended to cure, ameliorate, stabilize, or prevent a disease, pathological condition, or disorder, need not actually result in the cure, ameliorization, stabilization or prevention. The effects of treatment can be measured or assessed as described herein and as known in the art as is suitable for the disease, pathological condition, or disorder involved. Such measurements and assessments can be made in qualitative and/or quantitative terms. Thus, for example, characteristics or features of a disease, pathological condition, or disorder and/or symptoms of a disease, pathological condition, or disorder can be reduced to any effect or to any amount.

5. Optional

“Optional” or “optionally” or like terms means that the subsequently described event or circumstance can or cannot occur, and that the description includes instances where the event or circumstance occurs and instances where it does not. For example, the phrase “optionally the composition can comprise a combination” means that the composition may comprise a combination of different molecules or may not include a combination such that the description includes both the combination and the absence of the combination (i.e., individual members of the combination).

6. A

As used in the specification and the appended claims, the singular forms “a,” “an” and “the” or like terms include plural referents unless the context clearly dictates otherwise. Thus, for example, reference to “a pharmaceutical carrier” includes mixtures of two or more such carriers, and the like.

7. Abbreviations

Abbreviations, which are well known to one of ordinary skill in the art, may be used (e.g., “h” or “hr” for hour or hours, “g” or “gm” for gram(s), “mL” for milliliters, and “rt” for room temperature, “nm” for nanometers, “M” for molar, and like abbreviations).

8. Ranges

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

9. Comprise

Throughout the description and claims of this specification, the word “comprise” and variations of the word, such as “comprising” and “comprises,” means “including but not limited to,” and is not intended to exclude, for example, other additives, components, integers or steps.

10. Consisting Essentially of

“Consisting essentially of” in embodiments refers, for example, to a surface composition, a method of making or using a surface composition, formulation, or composition on the surface of the biosensor, and articles, devices, or apparatus of the disclosure, and can include the components or steps listed in the claim, plus other components or steps that do not materially affect the basic and novel properties of the compositions, articles, apparatus, and methods of making and use of the disclosure, such as particular reactants, particular additives or ingredients, a particular agents, a particular cell or cell line, a particular surface modifier or condition, a particular ligand candidate, or like structure, material, or process variable selected. Items that may materially affect the basic properties of the components or steps of the disclosure or may impart undesirable characteristics to the present disclosure include, for example, decreased affinity of the cell for the biosensor surface, aberrant affinity of a stimulus for a cell surface receptor or for an intracellular receptor, anomalous or contrary cell activity in response to a ligand candidate or like stimulus, and like characteristics.

11. Components

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these molecules may not be explicitly disclosed, each is specifically contemplated and described herein. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C—F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

12. Mimic

As used herein, “mimic” or like terms refers to performing one or more of the functions of a reference object. For example, a molecule mimic performs one or more of the functions of a molecule.

13. Or

The word “or” or like terms as used herein means any one member of a particular list and also includes any combination of members of that list.

14. Publications

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

15. Sample

By sample or like terms is meant an animal, a plant, a fungus, etc.; a natural product, a natural product extract, etc.; a tissue or organ from an animal; a cell (either within a subject, taken directly from a subject, or a cell maintained in culture or from a cultured cell line); a cell lysate (or lysate fraction) or cell extract; or a solution containing one or more molecules derived from a cell or cellular material (e.g. a polypeptide or nucleic acid), which is assayed as described herein. A sample may also be any body fluid or excretion (for example, but not limited to, blood, urine, stool, saliva, tears, bile) that contains cells or cell components.

16. About

About modifying, for example, the quantity of an ingredient in a composition, concentrations, volumes, process temperature, process time, yields, flow rates, pressures, and like values, and ranges thereof, employed in describing the embodiments of the disclosure, refers to variation in the numerical quantity that can occur, for example, through typical measuring and handling procedures used for making compounds, compositions, concentrates or use formulations; through inadvertent error in these procedures; through differences in the manufacture, source, or purity of starting materials or ingredients used to carry out the methods; and like considerations. The term “about” also encompasses amounts that differ due to aging of a composition or formulation with a particular initial concentration or mixture, and amounts that differ due to mixing or processing a composition or formulation with a particular initial concentration or mixture. Whether modified by the term “about” the claims appended hereto include equivalents to these quantities.

17. Values

Specific and preferred values disclosed for components, ingredients, additives, cell types, markers, and like aspects, and ranges thereof, are for illustration only; they do not exclude other defined values or other values within defined ranges. The compositions, apparatus, and methods of the disclosure include those having any value or any combination of the values, specific values, more specific values, and preferred values described herein.

Thus, the disclosed methods, compositions, articles, and machines, can be combined in a manner to comprise, consist of, or consist essentially of, the various components, steps, molecules, and composition, and the like, discussed herein.

18. Compounds and Compositions

Compounds and compositions have their standard meaning in the art. It is understood that wherever, a particular designation, such as a molecule, substance, marker, cell, or reagent compositions comprising, consisting of, and consisting essentially of these designations are disclosed.

19. Obtaining

Obtaining or like terms means getting or acquiring. For example, obtaining a sample includes taking a sample physically from a subject and it also includes receiving a sample which someone else took from a subject, which was for example, stored. Thus, obtaining includes but is not limited to physically collecting a sample.

20. Measuring the Length of the Chromosome Telomere

Measuring the length of the chromosome telomere or like terms includes directly measuring, such as by determining the number of bases or repeats or other physical ways of quantifying the absolute length of a telomere, as well as indirectly measuring the length. An indirect measurement of the length refers to measuring something which is a substitute or related or correlated to length, such as the amount of a telomere marker bound to a telomere, or the amount of signal arising from a telomere marker bound to a telomere, such as the amount of fluorescence bound to a telomere via a telomere marker.

It is understood that a single telomere or arm of a telomere can be measured up to and including all of the telomeres of each chromosome of a cell of a subject. There are 23 pairs of chromosomes, 46 individual chromosomes, and 92 arms of a chromosome having 92 telomeres in a typical human cell.

21. Chromosome 9p Telomere

Chromosome 9p telomere or like terms is the telomere on the short branch of chromosome 9.

22. Chromosome 9p Telomere Length

Chromosome 9p telomere length is the length of the short arm of chromosome 9.

23. Shorter

Shorter or like terms refers to fewer nucleotides. One nucleic acid, such as a chromosome or a telomere of a chromosome, would be shorter than another nucleic acid if it has at least one fewer nucleotide.

How much shorter a nucleic acid is than another nucleic acid can be represented by referring to the representative length of the two nucleic acids as less than or equal to 0.000001, 0.00001, 0.0001, 0.001, 0.01, 0.1, 0.2, 0.3, 0.4, 0.5, 0.6, 0.7, 0.8, 0.9, or 0.95 of the length of one or the other nucleic acid. For example, a nucleic acid that is 900 bases long is 0.9 the length of a nucleic acid that is 1000 bases long.

24. Chromosome 9p Reference Length

A chromosome 9p reference length or like terms is a reference length of the chromosome 9 short arm.

25. Relative Telomere Length or Telomere Ratio

A relative telomere length or telomere ratio or like terms is a ratio between the length of at least one telomere of one arm of a chromosome in a cell and the length of the reference nucleic acid sequences, such as the length of all the telomeres of a complete set of chromosome arms (N=92) in a typical human cell or the length of centromeric sequences of chromosome 2 etc. Understanding that length can be absolute length or an indirect measurement of length as discussed herein. Thus, a telomere ratio could be the signal from the short arm of chromosome 9p to the signal of a reference nucleic acid sequences, i.e. signals from the telomeres of the 92 arms of the chromosomes from a typical human cell. This would be a chromosome 9p relative telomere length or telomere ratio.

26. Reference Telomere Ratio

A reference telomere ratio or like terms is a telomere ratio that is produced from a sample(s) from a subject(s) that is considered a control. For example, it could be from healthy individuals or from non-cancerous patients. It is understood that the reference telomere ratio can be produced de novo or can be a number previously determined to be a reference number. As disclosed herein, a reference telomere ratio could be or be 0.00494, 0.00583, or 0.00680 or other like numbers, disclosed in tables 1-4, for example.

27. Increased Likelihood of Having Cancer or Contracting Cancer

Increased likelihood of having cancer or contracting cancer refers to an odds ratio as discussed herein. For example, it can be considered a fold likelihood relative to a group, such as a subject has at least a 3.0, 3.9, or 6.6, fold increase relative to all women, or at least a 2.1, 2.9, or 6.2, fold increase relative to all premenopausal women or at least a 4.3, 5.1, or 7.5, fold increase relative to all postmenopausal women.

28. Chromosome 9 Telomere Ratio

A chromosome 9 telomere ratio or like terms refers to a telomere ratio where the numerator of the telomere ratio is represented by the direct or indirect length of chromosome 9 telomeres.

29. Chromosome 9p Telomere Ratio

A chromosome 9p telomere ratio or like terms refers to a telomere ratio where the numerator of the telomere ratio is represented by the direct or indirect length of chromosome 9p telomeres.

30. Telomere Length

Telomere length or like terms refers to the direct or indirect length of a telomere of a chromosome arm.

31. Indirect Measurement

An indirect measurement or like terms refers to a measurement that is representative or something. For example, the amount of fluorescence signal arising from bound fluorescently labeled probe on a telomere or a chromosome is an indirect measurement of the length or the telomere of that chromosome.

32. Telomere Marker

A telomere marker or like terms is any molecule or substance that interacts preferentially with a telomere relative to another region of a chromosome. A telomere marker could be a hybridization probe for a telomere, such as a fluorescent probe.

33. Total Telomere Length

The total telomere length or like terms refers to the length, direct or indirect, or all of the telomeres in a cell.

34. Patient History

A patient or subject history or like terms refers to one or more items in the history of a subject which could be considered relevant to the subject, such as race, age, physical status, such as pre or post menopausal, smoking, alcohol consumption, family cancer history, or like items.

35. Adjusted Odds Ratio

An adjusted odds ratio or like terms refers to a odds ratio that has been adjusted, such as statistically adjusted, based on one or more characteristics obtained in a subject history from the subject the telomere ratio was obtained from.

36. Quantitating Telomere Length

Quantitating telomere length or like terms refers to measuring the length of the telomere and can be performed by for example by quantifying the fluorescence using TeloMeter, which is a program that is freely available from John Hopkins University website (internet site bui2.win.ad.jhu.edu/telometer/) or quantitating can arise from the commercially available Isis image software from Metasystems (web site www.metasystems.com/).

37. Fresh Whole Blood

Fresh whole blood refers to a sample of venous blood from a subject and was kept at 4° C. for less than 48 hours.

38. Preventive Treatment/Intervention for Cancer

Preventive treatment/intervention for cancer refer to the current preventive treatment regimes or protocols, that are designed to reduce the likelihood of getting cancer for a individual and are in use by physicians or health care organization. For example, Tamoxifen is subscribed for a women who are at high risk of breast cancer or bilateral surgically removal of ovaries are used to reduce the breast or ovarian cancer if a women is at very high risk of breast cancer, i.e., BRCA1 mutation carriers. Preventive treatment/intervention for cancer also refers to clinical protocols to monitor an individual who is at high risk of getting cancer more closely to detect a cancer early. Patient whose cancer is detected in a early stage usually has a better change of cure and survival.

B. Cancer

1. Breast Cancer

Breast cancer, like most human malignancies, is characterized by short or extremely long telomere in tumor cells and chromosomal instability (Baudis BMC Cancer 7:226, 2007; Shih et al. Cancer Res 61:818-822, 2001; Michor et al. Semin Cancer Biol 15:43-49, 2005). It is documented that chromosomal instability preferentially involves specific chromosome arms for each type of human cancer (Baudis BMC Cancer 7:226, 2007). In breast cancer, frequent chromosomal abnormalities in early stage breast tumors involves a few chromosomal arms, including gains of 1q, 8q, 17q, and 20q, and losses of 8p, 9p, 16q and 17p (Baudis BMC Cancer 7:226, 2007; Gorgoulis et al. Mol Med 4:807-822, 1998; An et al. Genes Chromosomes Cancer 17:14-20, 1996). Disclosed herein, chromosome arm-specific telomere deficiency was correlated with the cancer which is consistent with the underlying mechanisms for such arm-specific instability because critically short/dysfunctional telomeres can lead to chromosome end fusion which induces chromosome specific instability via repeated series of breakage-fusion-bridge (BFB) cycles (Hackett et al. Cell, 106:275-286, 2001; Gisselsson et al. PNAS 98:12683-12688, 2001; Stewenius et al. PNAS, 102:5541-5546, 2005; Lo et al. Neoplasia, 4:531-538, 2002).

Breast cancer, like most human malignancies, is characterized by chromosomal instability (CIN) (Baudis BMC Cancer 7:226, 2007). CIN is featured by losses or gains of entire chromosomes or chromosomal fragments, resulting in aneuploidy, large deletions or gains, and chromosomal rearrangements. CIN is observed as an early event in tumorigenesis (Shih et al. Cancer Res 61:818-822, 2001; Michor et al. Semin Cancer Biol 15:43-49, 2005) and there is abundant evidence of correlation between increasing chromosomal abnormalities and greater tumor aggressiveness. However, the molecular defects underlying CIN, and whether CIN is a cause or a consequence of the malignant phenotype, are not clear.

Accumulating evidence indicates that dysregulation of the p53 and Rb pathways are important mechanisms of CIN development (Hernando et al. Nature 430:797-802, 2004), and deregulation of the Rb pathway is a major contributor to CIN in breast tumors (Fridlyand et al. BMC Cancer 6:96, 2006; Gorgoulis et al. 4:807-822, 1998). One of the chromosomal abnormalities that affects the regulation of both p53 and Rb pathways is the chromosome 9p21 deletion. Indeed, deletions of chromosome 9 or 9p21 are the most frequent early chromosomal abnormalities in cancers, including head and neck (Miracca et al. 9:229-233, 2000), bladder (Mhawech-Fauceglia et al. Cancer 106:1205-1216, 2006), non-small cell lung (Sato et al. Genes Chromosomes Cancer 44:405-414, 2005) and skin cancers (Baudis et al. 2007; Rakosky et al. Cancer Genet Cytogenet 182:116-121, 2008). Frequent chromosome 9p deletions were also reported for high grade ductal carcinoma in situ (DCIS) (Ellsworth et al. Ann Surg Oncol 14:3070-3077, 2007; Hwang et al. Clin Cancer Res 10:5160-5167, 2004) and invasive breast cancer (An et al. Genes Chromosomes Cancer 17:14-20, 1996; Xie et al. Int J Oncol 21:499-507, 2002). On 9p, deletions and recombination are centered around an important tumor suppressor locus, the CDKN2A (Williamson et al. Hum Mol Genet. 4:1569-1577, 1995; Cairns et al. Nat Genet. 11:210-212, 1995). The CDKN2A gene encodes 2 proteins (p16INK4 and p14ARF) that regulate two critical cell cycle regulatory pathways: the p53 pathway and the retinoblastoma pathway (Harris et al. Oncogene 24:2899-2908, 2005). Thus inactivation of the CDKN2A locus via chromosome 9p21 deletion may be an initiating event in the development of breast cancer. Chromosomes possessing short telomeres may be unstable and so individuals who have short telomeres on chromosome 9 may have an increased likelihood of chromosome 9 deletion, and consequently a greater risk to develop cancer.

C. Telomeres

Telomeres are specialized DNA-protein structures that cap the ends of linear chromosomes. They are crucial for protecting linear chromosomes and are essential for maintaining the integrity and stability of genomes (McEachern et al. Annu Rev Genet, 34:331-358, 2000). Telomere-induced chromosomal instability could drive the tumorigenic process by increasing mutation rates for oncogenes and tumor suppressor genes (Maser et al. Science, 297:565-569, 2000).

Disclosed herein, a case-control study of breast cancer, examining the association between chromosome 9 arm-specific telomere lengths and breast cancer risk demonstrated that short telomere length on chromosome 9p is strongly associated with breast cancer risk, and provides new methods and compositions for breast cancer risk assessment for individuals in the general population.

D. Methods for Breast Cancer Assessment

One aspect of the present disclosure is directed to methods for detecting telomere shortening as diagnostic indicators of cancer risk, such as breast cancer. More particularly, in accordance with one embodiment methods are provided for detecting the presence of telomere shortening in the chromosomes prepared from blood cells of a subject. Being able to perform diagnostic tests for cancer risk, such as breast cancer, from blood cells, such as lymphocyte cells of the patient, is desirable in the field of cancer risk assessment. Disclosed is data showing that telomere lengths, such as chromosome 9P length, such as the 9P short arm length, have been associated with the risk of cancer and can serve as possible markers for cancer risk prediction.

Disclosed are methods of detecting the presence of shortened telomeres in blood cells is provided using a chromosome analysis based on quantitating telomere length disclosed herein. The method comprises the steps of obtaining blood from a subject, harvesting the chromosomes, performing chromosome analysis, quantitating telomere length and determining the cancer risk based on the length of the telomere.

Disclosed are methods of detecting the presence of shortened telomeres in blood cells is provided using a chromosome analysis based on quantitating telomere length disclosed herein. The method comprises the steps of obtaining blood from a subject, harvesting the chromosomes, performing telomere fluorescent in situ hybridization on chromosomes, quantitating telomere length and determining the cancer risk based on the length of the 9p telomere.

In one embodiment, the chromosome analysis is performed using telomere fluorescent in situ hybridization (FISH).

A significant advantage of the disclosed methods is that blood cells are used instead of tissue derived directly from breast or tumor. Therefore the test is much less invasive and can be used as pre-cancer screen as opposed to a post-cancer screen.

The results of the experiments discussed in the Examples, showed that, after adjustment for known breast cancer risk factors, shorter telomeres on chromosome 9p-short (allelic shorter version) were strongly associated with an increased risk of breast cancer, as described at least in Tables 1-4. This finding indicates that individuals who possess short telomeres on chromosome 9 are at increased risk of breast cancer.

In humans, there are 23 pairs of chromosomes and 92 telomeres, and chromosome specific telomere lengths are highly polymorphic between chromosomal arms (Lansdorp et al. Hum Mol Genet 5:685-691, 1996; Graakjaer et al. Mech Ageing Dev 124:629-640, 2003; Martens et al. Nat Genet 18:76-80, 1998). The disclosed observation that the shorter, such as the shortest, 9p telomere (shorter 9p-short telomeres) were strongly associated with breast cancer risk provides methods of using this information to assess the risk of cancer, such as breast cancer, in a subject. This study reports that a short telomere on chromosome 9p is strongly associated with breast cancer risk. Telomere length on chromosome 9p, as disclosed herein, is a tool for identifying women at risk for breast cancer and improving breast cancer risk assessment for individuals in general population.

Telomere lengths on chromosome 9q were not associated with breast cancer risk. No significant correlations was observed between 9p and 9q telomere lengths in controls, except a weak correlation between 9p-short and 9q-short, suggesting telomere lengths on 9p or 9q are independent events. There was no significant difference in mean overall (cell total) telomere length between cases and controls (Table 1) (Zheng et al. Breast Cancer Res Treat, in press, 2009). This may in part explain the null findings by three recent studies that examined the association of overall (cell total) telomere length in blood leucocytes and breast cancer risk (Shen et al. Cancer Res 67:5538-5544, 2007; Svenson et al. Cancer Res 68:3618-3623, 2008; Barwell et al. Br J Cancer 97:1696-1700, 2007).

Disclosed are methods of assaying a subject comprising, Obtaining a sample from the subject, Measuring the length of the chromosome 9p telomere in a cell of the sample, producing a chromosome 9p telomere length, and Identifying a subject having a chromosome 9p telomere length which is shorter than a chromosome 9p reference length or alone or in any combination.

Also disclosed are methods, wherein shorter comprises chromosome 9p telomere length less than or equal to 0.000001, 0.00001, 0.0001, 0.001, 0.01, 0.1, 0.2, 0.3, 0.4, 0.5, 0.6, 0.7, 0.8, 0.9, or 0.95 of the chromosome 9p reference length or alone or in any combination.

Disclosed are methods of assaying a subject comprising, Obtaining a sample from the subject, Measuring a telomere ratio from a cell from the sample, and Identifying a subject having a telomere ratio less than a reference telomere ratio or alone or in any combination.

Also disclosed are methods, wherein a subject having a telomere ratio less than a reference telomere ratio has an increased likelihood of having cancer, wherein a subject having a telomere ratio less than a reference telomere ratio has an increased likelihood of contracting cancer, wherein the likelihood indicates at least a 3.0, 3.9, or 6.6, fold increase relative to all women, wherein the likelihood indicates at least a 2.1, 2.9, or 6.2, fold increase relative to all premenopausal women, wherein the likelihood indicates at least a 4.3, 5.1, or 7.5, fold increase relative to all postmenopausal women, wherein the cancer is breast cancer, wherein the telomere ratio is a chromosome 9 telomere ratio, wherein the telomere ratio is a chromosome 9p telomere ratio, wherein the reference telomere ratio is 0.00494, 0.00583, or 0.00680, wherein measuring the telomere ratio comprises substituting an indirect measurement of telomere length represented in the telomere ratio, wherein measuring the telomere ratio comprises substituting an indirect measurement of telomere length for all telomeres represented in the telomere ratio, wherein indirect measurement comprises measuring a telomere marker, wherein the telomere marker comprises a telomere hybridization probe, wherein the hybridization probe comprises a fluorescent probe, wherein the indirect measurement comprises the fluorescent signal of a fluorescent in situ hybridization assay, wherein measuring the telomere ratio comprises the length of at least one telomere represented in the telomere ratio, wherein measuring the telomere ratio comprises the length of all telomeres represented in the telomere ratio, further comprising the step of measuring the length of all of the telomeres in the cell of the subject, producing a total telomere length, further comprising the step of comparing the length of the chromosome 9p in the cell of the subject to the length of all of the telomeres in the cell of the subject forming a telomere ratio, and/or further comprising the step of creating a telomere ratio, alone or in any combination.

Disclosed are methods of assaying a subject comprising, collecting a sample from the subject, measuring the length of the telomeres of the subject, creating a telomere ratio, wherein the 9p telomere ratio compares the length of the 9P telomere to the total length of the telomeres of all chromosomes of the subject, and identifying a subject having a 9p telomere ratio less than or equal to 0.00680 or alone or in any combination.

Also disclosed are methods, wherein the sample comprises a blood sample, wherein the 9P telomere length measured is the short arm, further comprising the step of identifying the subject as a subject having an increased risk of breast cancer, wherein the 9P telomere ratio is less than or equal to 0.00583, wherein the 9P telomere ratio is less than or equal to 0.00494, wherein collecting the sample comprises culturing lymphocytes within 48 hours after blood collection, wherein collecting the sample comprises harvesting the chromosomes with a cytogenic protocol, wherein measuring the length of the telomeres comprises using a fluorescent in situ hybridization assay, further comprising obtaining a patient history of the subject, further comprising adjusting the estimated cancer risk (odds ratio) based on one or more characteristics identified in the patient history, and/or wherein the odds ratio is analyzed, alone or in combination with other markers/host factors to predict cancer risk.

Disclosed are methods of assaying the risk of cancer, in a subject, based on telomere length comprising: taking a blood sample from the subject; harvesting the chromosomes; performing telomere analysis; quantitating telomere length; and determining the risk a subject of getting cancer if the subject's 9p telomere length is less than 0.680% or 0.583% or 0.494% of the length of all the subject's chromosomes telomere length alone or in combination with other markers/host factors.

Also disclosed are methods wherein cancer is breast cancer, wherein the chromosome 9p telomere is the shorter of the two 9p telomeres, 9p short, wherein telomere analysis comprises fluorescent in situ hybridization (FISH) analysis, wherein quantitating telomere length comprises quantifying the fluorescence using TeloMeter or Isis software.

Also disclosed are methods of treating a subject, such as a patient comprising, analyzing the results of the method of any of the methods disclosed herein, and performing a treatment treat or to prevent cancer on the subject based on the results of the method alone or in combination with other markers/host factors.

A method of assaying a subject comprising, collecting a sample from the subject and setting up a blood culture, performing a telomere fluorescent in situ hybridization, measuring the intensity of the telomere fluorescent signals of the hybridization from the sample of the subject, creating a telomere ratio, wherein the relative telomere length of the short arms of chromosome 9 (9p) is the ratio of the telomere signal intensity of the 9p to the telomere signal intensity of all the telomeres in a cell from the sample, and identifying a subject having a 9p telomere ratio less than or equal to a reference 9p telomere ratio or alone or in any combination with any other aspects disclosed herein.

Also disclosed are methods, wherein the sample comprises a blood sample, wherein the 9P telomere length measured is the short arm of chromosome #9, further comprising the step of identifying the subject as a subject having an increased risk of breast cancer, wherein the reference 9P telomere ratio is less than or equal to 0.680%, wherein the reference 9P telomere ratio is less than or equal to 0.583%, wherein the reference 9P telomere ratio is less than or equal to 0.494%, wherein setting up the lymphocyte culture within 48 hours of blood sample collection, wherein collecting the sample comprises culturing blood lymphocytes (see for example, Zheng et al, 2003, Carcinogenesis 24:269-74), wherein collecting the sample comprises harvesting the chromosomes with a cytogenic protocol (see for example, Zheng et al, 2003, Carcinogenesis 24:269-74), wherein measuring the length of the telomeres comprises using a fluorescence in situ hybridization (FISH) assay, wherein measuring the length of the telomeres comprises analyzing FISH images of metaphase chromosomes using the TeloMeter, further comprising obtaining a demographic, lifestyle and medical history of the subject, wherein the statistical analysis indicates at least a 3.0, 3.9, or 6.6, fold increase relative to all women, wherein the statistical analysis indicates at least a 2.1, 2.9, or 6.2, fold increase relative to all premenopausal women, and/or wherein the statistical analysis indicates at least a 4.3, 5.1, or 7.5, fold increase relative to all postmenopausal women, further comprising a statistical analysis (logistic regression modeling) to predict the likelihood of having breast cancer using the relative telomere length of chromosome 9p alone as the predictor or in combination with other markers/host factors to predict cancer risk alone or in any combination with any other aspects disclosed herein.

Disclosed are methods wherein relative to all women, the likelihood of having breast cancer given having shorter telomere length (0.583%-0.680%) on 9p increases 3.0-fold when compared to women having long telomere (>0.680%) on 9p, wherein relative to all women, wherein the likelihood of having breast cancer given having shorter telomere length (0.494%-0.583%) on 9p increases 3.9-fold when compared to women having long telomere (>0.680%) on 9p, wherein relative to all women, wherein the likelihood of having breast cancer given having shorter telomere length (<0.494%) on 9p increases 6.6-fold when compared to women having long telomere (>0.680%) on 9p, wherein relative to pre-menopausal women, wherein the likelihood of having breast cancer given having shorter telomere length (0.583%-0.680%) on 9p increases 2.1-fold when compared to women having long telomere (>0.680%) on 9p, wherein relative to pre-menopausal women, wherein the likelihood of having breast cancer given having shorter telomere length (0.494%-0.583%) on 9p increases 2.9-fold when compared to women having long telomere (>0.680%) on 9p, wherein relative to pre-menopausal women, wherein the likelihood of having breast cancer given having shorter telomere length (<0.494%) on 9p increases 6.2-fold when compared to women having long telomere (>0.680%) on 9p, wherein relative to post-menopausal women, wherein the likelihood of having breast cancer given having shorter telomere length (0.583%-0.680%) on 9p increases 4.3-fold when compared to women having long telomere (>0.680%) on 9p, wherein relative to post-menopausal women, wherein the likelihood of having breast cancer given having shorter telomere length (0.494%-0.583%) on 9p increases 5.1-fold when compared to women having long telomere (>0.680%) on 9p, and/or wherein relative to post-menopausal women, wherein the likelihood of having breast cancer given having shorter telomere length (<0.494%) on 9p increases 7.5-fold when compared to women having long telomere (>0.680%) on 9p alone or in any combination with any other aspects disclosed herein.

In certain embodiments, the methods address the relationship between length and cancer risk, such as breast cancer risk, that for every 10% decrease in relative telomere length of 9p, there is a 37% (95% CI=20%-57%, p<0.0001) increase in cancer risk, such as breast cancer risk. Thus, a step that can be added to any of the disclosed methods is a step of determining % decrease, or any equivalent operation, such as relative amounts, of the relative telomere length, or telomere ratio, and additionally, a step of converting this decrease to a risk assessment for breast cancer based on the disclosed relationship. Generally the idea of an increased risk of breast cancer based on a decrease in telomere length is disclosed.

Disclosed are methods of assaying the risk of cancer, in a subject, based on telomere length comprising: taking a blood sample from the subject; Setting up a lymphocyte culture using fresh whole blood; harvesting the chromosome preparation using standard cytogenetic method; performing quantitative telomere fluorescent in situ hybridization (FISH); analyzing FISH images of chromosome spreads to quantify telomere length (telomere signal intensity); defining the relative telomere length of chromosome 9p as the intensity of 9p telomere signal divided by the intensity of total telomere signals of the cell; determining the likelihood of getting cancer for a individual using statistic prediction model considering if the individual's 9p telomere length is less than a specific cut points (0.494%, 0.583% or 0.680%) or alone or in combination with other markers/host factors, alone or in any combination with any other aspects disclosed herein.

Also disclosed are methods, wherein cancer is breast cancer, wherein the chromosome 9p telomere is the shorter of the two 9p telomeres (9p-short), wherein the two of the chromosome 9p telomeres can be used jointly to predict cancer risk, wherein chromosome analysis comprises fluorescent in situ hybridization (FISH) analysis, wherein quantitating telomere length comprises quantifying the intensity of the fluorescence signal using TeloMeter or Isis image software or alone or in any combination.

Also disclosed are methods of treating a patient comprising, analyzing the results of the method of any of the methods disclosed herein alone or in combination with other markers/host factors to predict cancer risk, and based on the risk performing a preventive treatment/intervention for cancer on the subject.

1. Nucleic Acids

There are a variety of molecules disclosed herein that are nucleic acid based, as well as any other proteins disclosed herein, as well as various functional nucleic acids. The disclosed nucleic acids are made up of for example, nucleotides, nucleotide analogs, or nucleotide substitutes. Non-limiting examples of these and other molecules are discussed herein. It is understood that, for example, when a vector is expressed in a cell, the expressed mRNA will typically be made up of A, C, G, and U. Likewise, it is understood that if, for example, an antisense molecule is introduced into a cell or cell environment through, for example, exogenous delivery, it is advantageous that the antisense molecule be made up of nucleotide analogs that reduce the degradation of the antisense molecule in the cellular environment.

a) Nucleotides and Related Molecules

A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. A non-limiting example of a nucleotide would be 3′-AMP (3′-adenosine monophosphate) or 5′-GMP (5′-guanosine monophosphate).

A nucleotide analog is a nucleotide which contains some type of modification to the base, sugar, or phosphate moieties. Modifications to nucleotides are well known in the art and would include for example, 5-methylcytosine (5-me-C), 5-hydroxymethyl cytosine, xanthine, hypoxanthine, and 2-aminoadenine as well as modifications at the sugar or phosphate moieties.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid.

It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety. (Letsinger et al., Proc. Natl. Acad. Sci. USA, 1989, 86, 6553-6556),

A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

A Hoogsteen interaction is the interaction that takes place on the Hoogsteen face of a nucleotide or nucleotide analog, which is exposed in the major groove of duplex DNA. The Hoogsteen face includes the N7 position and reactive groups (NH2 or O) at the C6 position of purine nucleotides.

b) Sequences

There are a variety of sequences related to telomeres disclosed herein that are disclosed on Genbank, and these sequences and others are herein incorporated by reference in their entireties as well as for individual subsequences contained therein.

A variety of sequences are provided herein and these and others can be found in Genbank, at web site www.pubmed.gov. Those of skill in the art understand how to resolve sequence discrepancies and differences and to adjust the compositions and methods relating to a particular sequence to other related sequences. Primers and/or probes can be designed for any sequence given the information disclosed herein and known in the art.

c) Primers and Probes

Disclosed are compositions including primers and probes, which are capable of interacting with the genes disclosed herein. In certain embodiments the primers are used to support DNA amplification reactions. Typically the primers will be capable of being extended in a sequence specific manner. Extension of a primer in a sequence specific manner includes any methods wherein the sequence and/or composition of the nucleic acid molecule to which the primer is hybridized or otherwise associated directs or influences the composition or sequence of the product produced by the extension of the primer. Extension of the primer in a sequence specific manner therefore includes, but is not limited to, PCR, DNA sequencing, DNA extension, DNA polymerization, RNA transcription, or reverse transcription. Techniques and conditions that amplify the primer in a sequence specific manner are preferred. In certain embodiments the primers are used for the DNA amplification reactions, such as PCR or direct sequencing. It is understood that in certain embodiments the primers can also be extended using non-enzymatic techniques, where for example, the nucleotides or oligonucleotides used to extend the primer are modified such that they will chemically react to extend the primer in a sequence specific manner. Typically the disclosed primers hybridize with the nucleic acid or region of the nucleic acid or they hybridize with the complement of the nucleic acid or complement of a region of the nucleic acid.

E. Hybridization

The term hybridization typically means a sequence driven interaction between at least two nucleic acid molecules, such as a primer or a probe and a gene. Sequence driven interaction means an interaction that occurs between two nucleotides or nucleotide analogs or nucleotide derivatives in a nucleotide specific manner. For example, G interacting with C or A interacting with T are sequence driven interactions. Typically sequence driven interactions occur on the Watson-Crick face or Hoogsteen face of the nucleotide. The hybridization of two nucleic acids is affected by a number of conditions and parameters known to those of skill in the art. For example, the salt concentrations, pH, and temperature of the reaction all affect whether two nucleic acid molecules will hybridize.

Parameters for selective hybridization between two nucleic acid molecules are well known to those of skill in the art. For example, in some embodiments selective hybridization conditions can be defined as stringent hybridization conditions. For example, stringency of hybridization is controlled by both temperature and salt concentration of either or both of the hybridization and washing steps. For example, the conditions of hybridization to achieve selective hybridization can involve hybridization in high ionic strength solution (6×SSC or 6×SSPE) at a temperature that is about 12-25° C. below the Tm (the melting temperature at which half of the molecules dissociate from their hybridization partners) followed by washing at a combination of temperature and salt concentration chosen so that the washing temperature is about 5° C. to 20° C. below the Tm. The temperature and salt conditions are readily determined empirically in preliminary experiments in which samples of reference DNA immobilized on filters are hybridized to a labeled nucleic acid of interest and then washed under conditions of different stringencies. Hybridization temperatures are typically higher for DNA-RNA and RNA-RNA hybridizations. The conditions can be used as described above to achieve stringency, or as is known in the art. (Sambrook et al., Molecular Cloning: A Laboratory Manual, 2nd Ed., Cold Spring Harbor Laboratory, Cold Spring Harbor, N.Y., 1989; Kunkel et al. Methods Enzymol. 1987:154:367, 1987 which is herein incorporated by reference for material at least related to hybridization of nucleic acids). A preferable stringent hybridization condition for a DNA:DNA hybridization can be at about 68° C. (in aqueous solution) in 6×SSC or 6×SSPE followed by washing at 68° C. Stringency of hybridization and washing, if desired, can be reduced accordingly as the degree of complementarity desired is decreased, and further, depending upon the G-C or A-T richness of any area wherein variability is searched for. Likewise, stringency of hybridization and washing, if desired, can be increased accordingly as homology desired is increased, and further, depending upon the G-C or A-T richness of any area wherein high homology is desired, all as known in the art.

Another way to define selective hybridization is by looking at the amount (percentage) of one of the nucleic acids bound to the other nucleic acid. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the limiting nucleic acid is bound to the non-limiting nucleic acid. Typically, the non-limiting primer is in for example, 10 or 100 or 1000 fold excess. This type of assay can be performed at under conditions where both the limiting and non-limiting primer are for example, 10 fold or 100 fold or 1000 fold below their k_{d}, or where only one of the nucleic acid molecules is 10 fold or 100 fold or 1000 fold or where one or both nucleic acid molecules are above their k_{d}.

Another way to define selective hybridization is by looking at the percentage of primer that gets enzymatically manipulated under conditions where hybridization is required to promote the desired enzymatic manipulation. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer is enzymatically manipulated under conditions which promote the enzymatic manipulation, for example if the enzymatic manipulation is DNA extension, then selective hybridization conditions would be when at least about 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer molecules are extended. Preferred conditions also include those suggested by the manufacturer or indicated in the art as being appropriate for the enzyme performing the manipulation.

Just as with homology, it is understood that there are a variety of methods herein disclosed for determining the level of hybridization between two nucleic acid molecules. It is understood that these methods and conditions may provide different percentages of hybridization between two nucleic acid molecules, but unless otherwise indicated meeting the parameters of any of the methods would be sufficient. For example if 80% hybridization was required and as long as hybridization occurs within the required parameters in any one of these methods it is considered disclosed herein.

It is understood that those of skill in the art understand that if a composition or method meets any one of these criteria for determining hybridization either collectively or singly it is a composition or method that is disclosed herein.

F. Examples

1. Example 1

a) Results

(1) Characteristics of Study Population

Table 1 (shown below) lists the characteristics of the study subjects. The mean age is 52.7 for cases and 53.3 for controls. There are no significant case-control differences in the distributions of race, menopausal status, tobacco smoking, alcohol use, education levels, family income, history of pregnancy, and HRT use. The mean body mass index (BMI), mean age at first child birth, and mean age at menarche were almost identical between cases and controls. Controls were significantly more likely to be physically active in both the teens and in the past year compared with cases. There were no significant differences in the distribution of the levels of household income between cases and controls among those who reported household income. However, 31% of the cases and 16% of controls did not report household income (Table 1). Further experiments with a larger study population (N=205 for cases and N=236 for controls) showed a similar trend. Additional factors analyzed were age at menarche wherein the results showed 12.8+/−1.5 yrs for cases and 12.5+/−1.8 yrs for controls (p value of 0.583). Number of live births for cases was 1.53+/−1.31 and 1.51+/−1.29 yrs for controls (p value of 0.852). Age at first live birth was 27.1+/−6.3 yrs for cases and 28.8+/−6.6 for controls (p value 0.042)

TABLE 1
Distribution of Characteristics of Study Population

| | Cases | | Controls | | |
|---|---|---|---|---|---|
| Host factors | (N = 140) | | (N = 159) | | p-value |
| Age, mean (SD | 52.71 | (10.61) | 53.25 | (9.92) | 0.656 |
| Race, N (%) | | | | | |
| Whites | 104 | (74.2) | 118 | (74.2) | |
| Blacks | 28 | (20.0) | 34 | (21.4) | 0.850 |
| Others | 8 | (5.7) | 7 | (4.4) | 0.850 |
| Menopausal status, N (%) | | | | | |
| Pre- | 56 | (42.8) | 67 | (42.4) | |
| Post- | 75 | (57.3) | 91 | (57.6) | 0.953 |
| Tobacco smoking, N (%) | | | | | |
| Never | 81 | (60.0) | 93 | (58.9) | |
| Ever | 54 | (40.0) | 65 | (41.1) | 0.843 |
| Alcohol use, N (%) | | | | | |
| Never | 15 | (11.5) | 12 | (7.6) | |
| Ever | 116 | (88.6) | 147 | (92.5) | 0.225 |
| Physical Activity in | | | | | |
| teens, N (%) | | | | | |
| No | 55 | (39.3) | 31 | (19.5) | |
| Yes* | 85 | (60.7) | 128 | (80.5) | <0.001 |
| Physical activity last | | | | | |
| year, N (%) | | | | | |
| No | 59 | (42.1) | 36 | (22.6) | |
| Yes* | 81 | (57.9) | 123 | (77.4) | <0.001 |
| Education, N (%) | | | | | |
| <=4 years college | 78 | (58.2) | 82 | (51.6) | |
| >4 years college | 56 | (41.8) | 77 | (48.4) | 0.256 |
| Family income, N (%) | | | | | |
| <=100K | 46 | (32.9) | 56 | (42.1) | |
| >100K | 51 | (36.4) | 77 | (57.9) | |
| Missing | 43 | (30.7) | 26 | (16.4) | 0.010 |
| Family history of | | | | | |
| cancer\hat{ } | | | | | |
| No | 50 | (39.7) | 73 | (46.5) | |
| Yes | 76 | (60.3) | 84 | (53.5) | 0.250 |
| HRT§, N (%) | | | | | |
| No | 86 | (65.7) | 94 | (59.1) | |
| Yes | 45 | (34.4) | 65 | (40.9) | 0.254 |
| BMI†, mean (SD) | 27.14 | (6.30) | 27.28 | (6.64) | 0.862 |
| Total telomere length¶, | 4.36 | (0.99) | 4.58 | (1.01) | 0.069 |
| mean (SD) | | | | | |

*defined as physical activity at least once a week for at least 20 minutes at a time that either made the woman sweat or increased heart rate
\hat{ }defined as any cancer in the first or second degree blood relatives
§hormone replacement therapy
†body mass index
¶the unit of total telomere length is fluorescent intensity units in million (MFIU)

(2) Chromosome 9 Telomere Lengths by Case-Control Status

Table 2 presents the case-control comparisons of the means of the four chromosome 9 telomeres (9p-short, 9p-long, 9q-short, and 9q-long). The mean relative telomere length (RTL) of the 9p-short was significantly shorter in cases (mean=0.515%) than in controls (mean=0.593%, p<0.001). The mean RTL of the 9p-long was also significantly shorter in cases (mean=1.18%) than in controls (mean=1.234%, p=0.017). However, the mean RTLs of 9q-short and 9q-long were not significantly different between cases and controls. When the case-control comparison was stratified by race, menopausal status, tobacco smoking status, or physical activity in teens, we observed similar patterns of case-control differences across all subgroups of women.

TABLE 2
Case-Control Comparison of Mean Chromosome
9 Telomere Lengths, by Host Factors

| | Cases | | Controls | | |
|---|---|---|---|---|---|
| RTL (‰) | N | Mean (SD) | N | Mean (SD) | P-Value |

All subjects

| | | | | | | | |
|---|---|---|---|---|---|---|---|
| 9p-short TL | 140 | .515 | (.12) | 159 | .593 | (.12) | <0.001 |
| 9p-long TL | 140 | 1.184 | (.18) | 159 | 1.234 | (.17) | 0.017 |
| 9q-short TL | 140 | .512 | (1.23) | 159 | .525 | (.14) | 0.403 |
| 9q-long TL | 140 | 1.185 | (.9) | 159 | 1.176 | (.2) | 0.693 |

Pre-menopausal women

| | | | | | | | |
|---|---|---|---|---|---|---|---|
| 9p-short TL | 56 | .510 | (.13) | 67 | .604 | (.11) | <0.001 |
| 9p-long TL | 56 | 1.173 | (.180) | 67 | 1.244 | (.17) | 0.040 |
| 9q-short TL | 56 | .531 | (.11) | 67 | .529 | (.14) | 0.957 |
| 9q-long TL | 56 | 1.206 | (.20) | 67 | 1.162 | (.20) | 0.306 |

Post-menopausal women

| | | | | | | | |
|---|---|---|---|---|---|---|---|
| 9p-short TL | 75 | .516 | (.11) | 91 | .582 | (.13) | <0.001 |
| 9p-long TL | 75 | 1.189 | (.18) | 91 | 1.226 | (.18) | 0.267 |
| 9q-short TL | 75 | .504 | (.13) | 91 | .525 | (.14) | 0.214 |
| 9q-long TL | 75 | 1.173 | (.17) | 91 | 1.185 | (.19) | 0.486 |

Never smokers

| | | | | | | | |
|---|---|---|---|---|---|---|---|
| 9p-short TL | 81 | .514 | (.12) | 93 | .604 | (.12) | <0.001 |
| 9p-long TL | 81 | 1.191 | (.2) | 93 | 1.233 | (.16) | 0.132 |
| 9q-short TL | 81 | .505 | (.13) | 93 | .527 | (.14) | 0.259 |
| 9q-long TL | 81 | 1.190 | (.21) | 93 | 1.178 | (.2) | 0.711 |

Ever smokers

| | | | | | | | |
|---|---|---|---|---|---|---|---|
| 9p-short TL | 54 | .520 | (.11) | 65 | .575 | (.13) | 0.014 |
| 9p-long TL | 54 | 1.180 | (.17) | 65 | 1.238 | (.19) | 0.077 |
| 9q-short TL | 54 | .521 | (.12) | 65 | .522 | (.14) | 0.969 |
| 9q-long TL | 54 | 1.182 | (.16) | 65 | 1.177 | (.19) | 0.862 |

Women who were not physically active in their teens

| | | | | | | | |
|---|---|---|---|---|---|---|---|
| 9p-short TL | 55 | .490 | (.12) | 31 | .615 | (.13) | <0.001 |
| 9p-long TL | 55 | 1.174 | (.18) | 31 | 1.269 | (.15) | 0.011 |
| 9q-short TL | 55 | .513 | (.14) | 31 | .530 | (.15) | 0.601 |
| 9q-long TL | 55 | 1.202 | (.21) | 31 | 1.174 | (.19) | 0.522 |

Women who were physically active in their teens

| | | | | | | | |
|---|---|---|---|---|---|---|---|
| 9p-short TL | 85 | .531 | (.12) | 128 | .588 | (.13) | <0.001 |
| 9p-long TL | 85 | 1.191 | (.18) | 128 | 1.225 | (.18) | 0.174 |
| 9q-short TL | 85 | .512 | (.12) | 128 | .524 | (.14) | 0.494 |
| 9q-long TL | 85 | 1.174 | (.18) | 128 | 1.177 | (.20) | 0.914 |

RTL = relative telomere length

Later studies with a larger study population (N=204 for cases and 236 for controls) resulted in the following data. For all subjects, the mean RTL for cases was 0.663 and for controls was 0.692 (p value 0.0239). For pre-menopausal women, the mean RTL for cases was 0.665 and for controls was 0.719 (p value 0.0069). For post-menopausal women, the mean RTL for cases was 0.660 and for controls was 0.673 (p value 0.4449).

(3) RTL of Chromosome 9p-Short Telomere and Breast Cancer Risk

Table 3 shows the distributions of breast cancer patients and control subjects according to the RTL of 9p-short telomere. Using the 50th percentile value in controls as a cut point, women who had shorter 9p-short telomeres had significantly increased risk of breast cancer compared with women who had longer 9p-short telomeres (adjusted odds ratio [OR]=2.6; 95% confidence interval [CI]=1.5 to 4.3) in the overall study population. When stratified by menopausal status, the ORs were 2.8 (95% CI, 1.3 to 6.2) and 2.4 (95% CI, 1.2 to 4.9) for pre- and post-menopausal women, respectively. When the RTL data were categorized into quartiles, a significant inverse dose-response relationship was observed (P_{trend}<0.001), and the lowest-vs-highest quartile OR was 6.6 (95% CI, 2.8 to 15.9) for all women. The ORs were 6.2 (95% CI, 1.8 to 21.3) and 7.5 (95% CI, 2.1 to 26.8) for pre- and post-menopausal women, respectively.

TABLE 3
Logistic Regression Analysis Examining the Association of
Chromosome 9p-Short Telomere Length and Breast Cancer Risk

| 9p-short RTL | Case/Control | OR¹ | (95% CI) | P | OR² | (95% CI) | P |
|---|---|---|---|---|---|---|---|
| All Subjects | | | | | | | |
| ≧median (≧5.83) | 41/81 | 1.00 | | | 1.00 | | |
| <median (<5.83) | 99/78 | 2.53 | (1.56-4.12) | <0.001 | 2.58 | (1.54-4.33) | <0.001 |
| Long (≧6.80) | 10/41 | 1.00 | | 1.00 | | | |
| Short (<6.80) | 130/118 | 5.08 | (2.35-10.96) | <0.001 | 4.43 | (1.98-9.88) | |
| By quartiles | | | | | | | |
| Q4 (≧6.80) | 10/41 | 1.00 | | | 1.00 | | |
| Q3 (5.83-6.80) | 31/40 | 3.4. | (1.44-8.19) | | 3.00 | (1.20-7.45) | |
| Q2 (4.94-5.83) | 35/40 | 3.93 | (1.66-9.29) | | 3.87 | (1.58-9.49) | |
| Q1 (≦4.94) | 64/38 | 7.51 | (3.25-17.37) | <0.001* | 6.62 | (2.75-15.94) | 0.001* |
| Pre-menopausal women | | | | | | | |
| ≧median (≧5.83) | 17/38 | 1.00 | | | 1.00 | | |
| <median (<5.83) | 39/29 | 3.00 | (1.42-6.35) | 0.004 | 2.80 | (1.28-6.16) | 0.010 |
| Long (≧6.80) | 5/18 | 1.00 | | | 1.00 | | |
| Short (<6.80) | 51/49 | 3.91 | (1.34-11.45) | 0.013 | 3.62 | (1.18-11.08) | 0.024 |
| By quartiles | | | | | | | |
| Q4 (≧6.80) | 5/18 | 1.00 | | | 1.00 | | |
| Q3 (5.83-6.80) | 12/20 | 2.29 | (0.67-7.85) | | 2.16 | (0.60-7.83) | |
| Q2 (4.94-5.83) | 11/14 | 2.90 | (0.81-10.35) | | 2.87 | (0.76-10.81) | |
| Q1 (≦4.94) | 28/15 | 6.99 | (2.14-22.83 | <0.001* | 6.16 | (1.78-21.32) | 0.002* |
| Post-menopausal women | | | | | | | |
| ≧median (≧5.83) | 22/42 | 1.00 | | | 1.00 | | |
| <median (<5.83) | 53/47 | 2.08 | (1.09-3.99) | 0.027 | 2.41 | (1.20-4.87) | 0.014 |
| Long (≧6.80) | 4/22 | 1.00 | | | 1.00 | | |
| Short (<6.80) | 71/67 | 5.89 | (1.91-18.14) | 0.002 | 5.66 | (1.75-18.28) | 0.004 |
| By quartiles | | | | | | | |
| Q4 (≧6.80) | 4/22 | 1.00 | | | 1.00 | | |
| Q3 (5.83-6.80) | 18/20 | 5.21 | (1.49-18.18) | | 4.32 | (1.15-16.26) | |
| Q2 (4.94-5.83) | 21/26 | 4.60 | (1.36-15.49) | | 5.13 | (1.45-18.13) | |
| Q1 (≦4.94) | 32/23 | 8.24 | (2.46-27.66) | 0.002* | 7.53 | (2.11-26.79) | 0.003* |

*P-for-trend
OR¹ adjusted for age as continuous and race (when appropriate).
OR² adjusted for age as continuous, race (when appropriate), smoking status (never/ever), alcohol use (never/ever), education, family history of cancer (no/yes), hormone replacement therapy (no/yes), history of pregnancy (no/yes), menopausal status (when appropriate), physical activity in teens (no/yes).

Later studies with a larger study population (N=440 for all subjects, N=185 for pre-menopausal women and N=242 for post-menopausal women) resulted in the following data. The OR for all subjects by median was 1.13 (0.75-1.69) (p value 0.5632); by quartiles was for Q3 2.18 (1.21-3.91), Q2 1.55 (0.84-2.84) and for Q1 1.88 (1.04-3.41) which had a p value of 0.1400. The OR for pre-menopausal women by median was 1.42 (0.75-2.69) (p value 0.2764); by quartiles was for Q3 3.14 (1.28-7.72), Q2 2.23 (0.89-5.61) and for Q1 2.54 (1.00-6.43) which had a p value of 0.0860. The OR for post-menopausal women by median was 1.02 (0.59-1.79) (p value 0.9366); by quartiles was for Q3 1.55 (0.68-3.54), Q2 1.27 (0.54-2.98) and for Q1 1.37 (0.61-3.08) which had a p value of 0.6470. The OR for this study was adjusted for age, race education, household income, physical activity in teens, smoking status, alcohol use, family history or cancer and history of pregnancy and shown in the parenthesis is the 95% CI).

(4) RTL of Chromosome 9p-Long Telomere and Breast Cancer Risk

Table 4 shows the distributions of breast cancer patients and control subjects according to the RTL of 9p-long telomere. Using the 50ₜₕ value in controls as a cut point, women who had shorter 9p-long telomere had marginally significant increased risk of breast cancer compared with women who had longer 9p-long telomere (OR=1.6, 95% CI=1.0 to 2.7) in the overall study population. When the RTL data were categorized into quartiles, a significant inverse dose-response relationship was observed (P_{trend}=0.020), and the lowest-vs-highest quartile odds ratio was 2.2 (95% CI, 1.1 to 4.4) for all women. The odds ratios were 3.0 (95% CI, 1.1 to 8.7) and 1.6 (95% CI, 0.6 to 4.3) for pre- and post-menopausal women, respectively.

TABLE 4
Logistic Regression Analysis Examining the Association of
Chromosome 9p-Long Telomere Length and Breast Cancer Risk

| 9p-long RTL | Case/Control | OR¹ | (95% CI) | P | OR² | (95% CI) | P |
|---|---|---|---|---|---|---|---|
| All Subjects | | | | | | | |
| ≧median (≧12.11) | 54/81 | 1.00 | | | 1.00 | | |
| <median (<12.11) | 86/87 | 1.65 | (1.03-2.64) | 0.037 | 1.63 | (0.99-2.67) | 0.056 |
| Long (≧13.43) | 24/40 | 1.00 | | 1.00 | | | |
| Short (<13.43) | 116/119 | 1.56 | (0.89-2.796) | 0.118 | 1.56 | (0.83-2.79) | 0.170 |
| By quartiles | | | | | | | |
| Q4 (≧13.43) | 24/40 | 1.00 | | | 1.00 | | |
| Q3 (12.11-13.43) | 30/41 | 1.18 | (0.59-2.36) | | 1.12 | (0.54-2.36) | |
| Q2 (11.22-12.11) | 35/39 | 1.47 | (0.73-2.94) | | 1.27 | (0.60-2.66) | |
| Q1 (≦11.22) | 51/39 | 2.11 | (1.09-4.07) | 0.018* | 2.19 | (1.09-4.39) | 0.020* |
| Pre-menopausal women | | | | | | | |
| ≧median (≧12.11) | 21/36 | 1.00 | | | 1.00 | | |
| <median (<12.11) | 35/31 | 1.97 | (0.95-4.12) | 0.070 | 1.84 | (0.84-4.00) | 0.126 |
| Long (≧13.43) | 10/20 | 1.00 | | | 1.00 | | |
| Short (<13.43) | 46/47 | 2.00 | (0.84-4.77) | 0.117 | 1.84 | (0.74-4.60) | 0.192 |
| By quartiles | | | | | | | |
| Q4 (≧13.43) | 10/20 | 1.00 | | | 1.00 | | |
| Q3 (12.11-13.43) | 11/16 | 1.41 | (0.48-4.19) | | 1.32 | (0.42-4.19) | |
| Q2 (11.22-12.11) | 12/16 | 1.52 | (0.52-4.47) | | 1.18 | (0.36-3.81) | |
| Q1 (≦11.22) | 23/15 | 3.18 | (1.16-8.74) | 0.025* | 3.03 | (1.06-8.74) | 0.042* |
| Post-menopausal women | | | | | | | |
| ≧median (≧12.11) | 31/44 | 1.00 | | | 1.00 | | |
| <median (<12.11) | 44/47 | 1.35 | (0.72-252) | 0.345 | 1.45 | (0.75-2.80) | 0.275 |
| Long (≧13.43) | 13/20 | 1.00 | | | 1.00 | | |
| Short (<13.43) | 62/71 | 1.36 | (0.60-2.96) | 0.446 | 1.26 | (0.55-2.88) | 0.586 |
| By quartiles | | | | | | | |
| Q4 (≧13.43) | 13/20 | 1.00 | | | 1.00 | | |
| Q3 (12.11-13.43) | 18/24 | 1.15 | (0.45-2.93) | | 0.96 | (0.35-2.60) | |
| Q2 (11.22-12.11) | 19/23 | 1.29 | (0.50-3.31) | | 1.17 | (0.43-3.20) | |
| Q1 (≦11.22) | 25/24 | 1.61 | (0.66-3.96) | 0.270* | 1.64 | (0.63-4.26) | 0.233* |

*P-for-trend
OR¹ adjusted for age as continuous and race (when appropriate).
OR² adjusted for age as continuous, race (when appropriate), smoking status (never/ever), alcohol use (never/ever), education, family history of cancer (no/yes), hormone replacement therapy (no/yes), history of pregnancy (no/yes), menopausal status (when appropriate), physical activity in teens (no/yes).

Table 5 presents the joint effects of 9p-short and 9p-long RTL on breast cancer risk. The data suggested additive effects. However, no statistical significant interactions between 9p-short and 9p-long RTL were detected in all subjects (P=0.108), pre-menopausal women (P=0.113) or post-menopausal women (P=0.415) when the interactions were formally tested in logistic models.

TABLE 5
Joint effect of Chromosome 9p-Short and 9p-
Long Telomere Length on Breast Cancer Risk

| | | | |
|---|---|---|---|
| 9p-short | 9p-long | Case/Control | OR (95% CI) |

All Subjects

| | | | |
|---|---|---|---|
| Long* | Long* | 30/53 | 1.00 |
| Short | Long | 24/28 | 2.71 (1.45-5.04) |
| Long | Short | 11/28 | 1.73 (0.85-3.54) |
| Short | Short | 75/50 | 3.97 (1.71-9.19) |

Pre-menopausal women

| | | | |
|---|---|---|---|
| Long | Long | 13/25 | 1.00 |
| Short | Long | 8/11 | 3.05 (1.20-7.78) |
| Long | Short | 4/13 | 2.44 (0.73-8.24) |
| Short | Short | 31/18 | 5.49 (1.46-20.7) |

Post-menopausal women

| | | | |
|---|---|---|---|
| Long | Long | 16/27 | 1.00 |
| Short | Long | 15/17 | 2.44 (1.04-5.71) |
| Long | Short | 6/15 | 1.39 (0.56-3.45) |
| Short | Short | 38/32 | 3.25 (1.08-9.82) |

*Chromosome 9p-short and 9p-long RTL were dichotomized by the median value in controls.
Ors were adjusted for age as continuous, race, smoking status (never/ever), alcohol use (never/ever), education, family history of cancer (no/yes), hormone replacement therapy (no/yes), history of pregnancy (no/yes), menopausal status (when appropriate), physical activity in teens (no/yes).
No significant interactions between 9p-short and 9p-long were detected.

(5) RTL of Chromosome 9q-Short and 9q-Long Telomeres and Breast Cancer Risk:

Using the 50th percentile value in controls as a cut point, when women who had shorter 9q-short telomere were compared with women had longer 9q-short telomere, the OR was 1.0 (95% CI, 0.6 to 1.7) for all women. When stratified by menopausal status, the ORs were 1.4 (95% CI, 0.7 to 2.7) and 0.8 (95% CI, 0.4 to 1.8) for pre- and post-menopausal women, respectively. When the data were categorized into quartiles, no significant dose-response relationship or a statistically significant difference by comparing the lowest to highest quartile was observed.

Similarly, when we used the 50th percentile value in controls as a cut point, women who had shorter 9q-long telomeres compared to longer 9q-long telomeres had an adjusted OR of 1.3 (95% CI, 0.8 to 2.0) overall. When stratified by menopausal status, the ORs were 1.6 (95% CI, 0.8 to 3.0) and 1.0 (95% CI, 0.5 to 2.1) for pre- and post-menopausal women, respectively. When the data were categorized into quartiles, no significant dose-response relationship or a statistically significant difference by comparing the lowest to highest quartile was observed.

(6) Correlations of Chromosome 9 Telomere Lengths in Controls:

Among control subjects, a weak inverse correlation was observed between 9p-short telomere length and age [Pearson correlation coefficient (r)=−0.162, P=0.042]. No significant correlations were seen between age and the other three chromosome 9 telomeres. There were significant correlations between the two 9p telomere lengths (r=0.431, P<0.001), and between the two 9q telomere lengths (r=0.428, P<0.001). There were no significant correlations between 9p telomeres and 9q telomere lengths, except for a weak correlation between 9p-short and 9q-short (r=0.204, P=0.010).

Among cases, there were no significant correlations between chromosome 9 telomere lengths and age. There were significant correlations between the two 9p telomeres (r=0.525, P<0.001), and between the two 9q telomeres (r=0.326, P<0.001). There were statistically significant but weak correlations between 9p-short and 9q-short (r=0.256, P=0.002), and between 9p-long and 9q-long (r=0.186, P=0.028).

(7) Telomere Length Variation Between Chromosome 9 Telomeres and Breast Cancer Risk

Increased telomere length variations across the genome increase genomic instability resulting in an increased risk of breast cancer, thus the differences in length between homologous chromosome 9 telomeres were examined for an association with breast cancer risk. Homologous telomere length difference (HTLD) was defined as the percent of (chromosome 9 long RTL−chromosome 9 short RTL) divided by (chromosome 9 long RTL+chromosome 9 short RTL). Initial case-control comparison of mean HTLD identified chromosome arm 9p as showing significant case-control difference at p-value <0.001 level (significant after Bonferroni correction for multiple comparisons 0.05/46=0.0011) in pre-menopausal women. The HTLD for all subjects in the case group (N=204) was 38.91 and the control group (N=236) was 37.14 (p value 0.0169). The HTLD for pre-menopausal women in the case group (N=89) was 39.10 and for controls (N=96) was 35.90 (p value 0.0005. The HTLD for post-menopausal women in the case group (N=110) was 38.95 and for controls (N=132) was 38.11 (p value 0.6494). The Wilcoxan rank sum test was used to calculate p values for HTLD study.

Using the 50^{th} percentile value in controls as a cut point, multivariate logistic regression analysis confirmed that greater difference in length between homologous telomeres on chromosome arm 9p was significantly associated with an increased breast cancer risk in premenopausal women (N=185), adjusted odds ratio (OR)=4.6 (95% CI=2.3 to 9.2) significant p value of <0.0001. The OR for all subjects (N=440) was 1.92 (95% CI=1.26-2.92), p value 0.0022, and for post-menopausal women (N=242) was 1.07 (95% CI=0.61-1.88), p value 0.8262. When the study subjects were categorized into four groups (by quartiles) according to the telomere length, a significant dose-response relationship was observed for chromosome 9p (P_{trend}<0.001). For all subjects, the OR for Q2 was 0.94 (95% CI=0.55-1.62), Q3 was 1.82 (95% CI=1.01-3.28) and Q4 was 1.98 (1.09-3.58) with a p value of 0.0052. For pre-menopausal subjects, the OR for Q2 was 1.22 (95% CI=0.46-3.21), Q3 was 7.18 (95% CI=2.48-20.79) and Q4 was 4.29 (1.53-11.99) with a p value of 0.0002. For post-menopausal subjects, the OR for Q2 was 1.03 (95% CI=0.50-2.12), Q3 was 0.83 (95% CI=0.38-1.80) and Q4 was 1.45 (0.65-3.16) with a p value of 0.5411. Chromosome 9p did not show a significant association with breast cancer risk in post-menopausal women.

b) Methods

(1) Study Population

Breast cancer cases were recruited at the Georgetown University Hospital clinics (Lombardi Comprehensive Cancer Center's Division of Medical Oncology, Department of Surgery and the Betty Lou Ourisman Breast Cancer Clinic). The inclusion criteria for cases included a diagnosis of breast cancer within the prior 6 months, women, have not been treated yet with chemotherapy and radiotherapy, ability to provide informed consent in English. Exclusion criteria were women with a prior history of cancer, had chemotherapy and radiation treatment, or had active infection or immunological disorder that needed to be treated with antibiotics or immunosuppressive medication within the prior one month. From 2006 through 2008, a total of 228 newly diagnosed breast cancer patients were identified to be eligible and 153 (67%) participated in the study. The common reasons for non-participation were: too busy or not interested (24%), overwhelmed by cancer diagnosis (5%), and not responsive to phone call or e-mail contact (4%).

Controls (N=159) were randomly selected from healthy women who visited the mammography screening clinic at Georgetown University Hospital, frequency matched to cases by age (2-year interval), race, and state of residency (D.C., Maryland or Virginia). Other inclusion and exclusion criteria for controls were the same as for cases. Additionally, women who had breast biopsy or were pregnant or breast feeding were not eligible. The overall participation rate among the eligible women was 57% for controls. The major reason for non-participation was being either too busy (19%) or not interested (23%).

After providing informed consent, subjects received a structured, in-person interview assessing prior medical history, tobacco smoke exposures, alcohol use, current medications, family medical history, reproductive history, and socioeconomic characteristics. Venous blood was obtained by trained interviewers using heparinized tubes. The study was approved by the MedStar Research Institute-Georgetown University Oncology Institutional Review Board.

(2) Blood Cultures and Preparation of Chromosome Spreads

Lymphocyte cultures were prepared from fresh blood within 48 hours after the samples were obtained, as previously described (Zheng et al. Cancer Res 65:9466-9573, 2005). One ml of fresh whole blood was added to 9 ml of RPMI-1640 medium supplemented with 15% fetal bovine serum (Biofluid, Inc. Rockville), 1.5% of phytohemagglutinin (Invitrogen Co., Carlsbad, Calif.), 2 mM L-glutamine, and 100 U/ml each of penicillin and streptomycin. Cells were cultured for 4 days at 37° C. and colcemid (0.2 μg/ml) was added to the culture 1 hour before the harvest. The cells were treated in hypotonic solution (0.06 M KCl) at room temperature (RT) for 25 mins, fixed in crayon fixative (methanol:acetic acid=3:1), and kept in crayon fixative at −20° C.

(3) Telomere Quantitative Fluorescent In Situ Hybridization (FISH)

The chromosome preparations were dropped onto clean microscopic slides and kept at room temperature for 7 days. Slides were then fixed in crayon fixative for one hour, dehydrated through an ethanol series (70%, 80%, 90% and 100%), and air dried. Fifteen microliters of hybridization mixture consisting of 0.3 μg/ml Cy3-labeled telomere-specific peptide nucleic acid (PNA) probe, 30 ng/ml of FITC-labeled chromosome 9-specific PNA probe, 50% formamide, 10 mM Tris-HCl, pH 7.5, 5% blocking reagent, and 1×Denhart's solution was applied to each slide. Slides were then placed in a Hybex microarray hybridization system where the DNA was denatured by incubating at 75° C. for five minutes, followed by hybridizing at 30° C. for three hours. After hybridization, the slides were sequentially washed; once in 1×SSC, once in 0.5×SSC, and once in 0.1×SSC; each wash was 10 min at 42° C. The slides were then mounted in anti-fade mounting medium containing 300 ng/ml 4′-6-diamidino-2-phenylindole (DAPI).

The slides were analyzed using an epifluorescence microscope equipped with a charge-coupled device (CCD) camera. Images were captured with exposure times of 0.15, 0.25 and 0.05 second for Cy3, FITC and DAR signals, respectively. Digitized images were quantified using a semiautomated script, TeloMeter (provided by Dr. Alan Meeker), which was written with image analysis software (ImageJ). This software permits measurement of telomere signals in defined regions, i.e., single chromosome arm or a cell (Meeker et al. Am J Pathol 164:925-935, 2004). Telomere length was expressed as fluorescent intensity units (FIU). Between the pair of chromosome 9 short arms (9p) or long arms (9q), one telomere is always shorter than the other and there are significant differences in lengths between allelic telomeres. This observation is consistent with previous reports indicated that arm-specific telomere lengths were highly variable between chromosome arms and between allelic arms (Gilson et al. Cell Cycle, 6:2486-2494, 2007; Graakjaer et al. Hum. Genet., 119:344-350, 2006). Thus, 4 telomeres from chromosome 9 were recorded separately and treated as separate parameters. For each patient, 15 metaphase spreads were analyzed to estimate the mean telomere length for the: (i) short version of chromosome 9p (9p-short); (ii) long version of chromosome 9p (9p-long); (iii) short version of chromosome 9q (9q-short); (iv) long version of chromosome 9q (9q-long); and (v) total telomere length of the cell. The relative telomere lengths (RTL) of chromosome 9 arms were defined as the ratio of the arm-specific telomere FIU to the total telomere FIU of the cell, thus effectively normalizing the hybridization variations among individual samples. The rationale to use total telomere signals as reference signal for the normalization are: (i) the same probe was used to measure total telomere length as arm-specific telomere length, thus providing a most efficient normalization and (ii) total telomere length is not significantly associated with breast cancer risk in this study (Zheng et al. Breast Cancer Res Treat, in press, 2009), which is also consistent with previous reports (Shen et al. Cancer Res, 67:5538-5544, 2007; Barwell et al. Br. J. Cancer, 97:1696-1700, 2007; De, et al. Cancer Epidemiol. Biomarkers Prev., 18:1152-1156, 2009).

Several quality control steps were used in this assay. Laboratory personnel who were responsible for the blood culture and telomere assay were blinded to the case-control status of the subjects. All new lots of reagents were tested to ensure optimal hybridization. A control slide containing cells with known total telomere length was included in each batch of TQ-FISH to monitor the quality of the hybridization efficiency. In this study, the coefficient of variation (CV) of total telomere length from 20 repeats of the control slide was 12.4%.

(4) Statistical Analysis

The final sample size for case-control analysis is at least 140 cases and 159 controls. Eleven cases were excluded due to no histological confirmation of breast cancer diagnosis (N=8) and blood culture failure (N=5). Student t-test was used to compare the means of chromosome 9 telomere lengths between cases and controls because the normality test indicated that these variables were symmetrically distributed. Chi-square tests were used to compare the distribution of categorical variables between cases and controls. Pearson correlation was used to examine the correlations between chromosome 9 telomere lengths and age.

The associations between chromosome 9 telomere lengths and the risk of breast cancer, using unconditional logistic regression, were examined. Telomere lengths were dichotomized as short/long using the 50^{th} or 75^{th} percentile values in the controls as a cut point. Telomere lengths were also categorized according to the quartiles in controls. Odds ratios were adjusted for age, race, smoking status, alcohol use, education, history of cancer in 1^{st} or 2^{nd} degree relatives, menopausal status, physical activity in the teens. P-values were two-sided and considered statistically significant if P<0.05. All analyses were performed using SAS software, version 9 (SAS Institute Inc., Cary, N.C.).

The case-control difference was unlikely to be ascribed to assay bias, because the samples were processed and measured blinded to case-control status. Measurements of four chromosome 9 arm telomere lengths were made simultaneously from the same cells captured by the fluorescent imaging system. While there were significant case-control differences for chromosome 9p telomere lengths, the mean telomere lengths of chromosome 9q and cell total were very similar between cases and controls. During the study, a systematic quality control plan was implemented to ensure the consistent efficiency of FISH hybridization (the CV of the total telomere lengths among 20 repeats of the control cell=12.4%). Co-hybridization of a FITC-labeled chromosome 9 specific probe (green) was used as an internal control for hybridization efficiency and only slides showed bright green chromosome 9 signals were accepted. The slides that failed to meet these quality control standards were rejected and repeated (3% of the slides). Additionally, relative telomere lengths of chromosome 9 arms were defined as the ratio of chromosome 9 telomere lengths to the total telomere length, thus minimizing the assay variation between the individual slides. Bias in telomere length measurement is thus unlikely.

Given that this is a case-control study, a theoretical concern is that telomere length in leucocytes is affected by case status (reverse causality). Data by previous studies and by us indicated that mean overall telomere length of blood leucocytes in breast cancer patients was not significantly shorter than in healthy women controls (Shen et al. Cancer Res 2007; Svenson et al. Cancer Res 2008; Barwell et al. Br J Cancer 2007), suggesting there is no significant shortening of blood leucocyte telomere length associated with having breast cancer. Although previous studies (Schroder et al. Br J Cancer 84:1348-1353, 2001; Yoon et al. Acta Haematol 118:30-37, 2007) indicated that chemotherapy and/or radiotherapy can induce telomere shortening in blood leucocytes, all the blood samples in this study were drawn before any chemotherapy and radiotherapy treatments. Thus reverse causality is not a plausible explanation for our results.

Recall bias might influence information about self-reported breast cancer risk factors in a case-control study where the data were collected after the diagnosis of cancer. Chromosome 9 telomere lengths were compared in control subjects by numerous variables from the questionnaire and no significant differences were found in the mean chromosome 9 telomere lengths between subgroups defined by race, age, smoking status, alcohol drinking, menopausal status, physical activity in the teens, hormone replacement therapy, history of pregnancy, family history of cancer, education, and income (data not shown). Cases and controls were closely matched by age, and age was included as continuous variable in all the logistic models for adjustment. Thus age would not confound the telomere results.

G. Example 2

Correlation Analysis of Breast Cancer Risk with Relative Telomere Length, Homologous Telomere Length Difference, and within-Cell Telomere Length Variation

This example describes the first genome-wide telomere association study to examine the associations between lengths of 92 telomeres in blood lymphocytes and breast cancer risk. The correlations discovered indicate roles of chromosomal telomeres in breast cancer susceptibility and provide the foundation of the disclosed methods.

1. Methods

a) Study Population

The study was approved by the MedStar Research Institute-Georgetown University Oncology Institutional Review Board. The details of study population were described previously (Zheng, 2009). Breast cancer cases (N=204) were recruited at the Georgetown University Medical Center clinics (Lombardi Comprehensive Cancer Center's Division of Medical Oncology, Department of Surgery and the Betty Lou Ourisman Breast Cancer Clinic). The inclusion criteria for cases included a diagnosis of breast cancer within the prior 6 months, women, have not been treated yet with chemotherapy and radiotherapy, ability to provide informed consent in English. Exclusion criteria were women with a prior history of cancer, had chemotherapy and radiation treatment, or had active infection or immunological disorder that needed to be treated with antibiotics or immunosuppressive medication within the prior one month. The overall participation rate among eligible patients is 70%.

Controls (N=236) were randomly selected from healthy women who visited the mammography screening clinic at Georgetown University Medical Center, frequency matched to cases by age (2-year interval), race, and state of residency (D.C., Maryland or Virginia). Other inclusion and exclusion criteria for controls were the same as for cases. Additionally, women who had breast biopsy or were pregnant or breast feeding were not eligible. The overall participation rate among the eligible women was 60% for controls. After providing informed consent, subjects received a structured, in-person interview assessing prior medical history, tobacco smoke exposures, alcohol use, current medications, family medical history, reproductive history, and socioeconomic characteristics. Venous blood was obtained by trained interviewers using heparinized tubes.

b) Chromosome Preparation from Blood Cultures

Short-term lymphocyte cultures were established from fresh blood within 48 hours after the samples were obtained, as previously described (Zheng, 2005). One ml of fresh whole blood was added to 9 ml of RPMI-1640 medium supplemented with 15% fetal bovine serum, 1.5% of phytohemagglutinin, 2 mM L-glutamine, and 100 U/ml each of penicillin and streptomycin. Cells were cultured for 4 days at 37° C. and colcemid (0.2 μg/ml) was added to the culture 1 hour before the harvest. The cells were treated in hypotonic solution (0.06 M KCl) at room temperature (RT) for 25 mins, fixed in crayon fixative (methanol:acetic acid=3:1), and kept in crayon fixative at −20° C.

c) Telomere Length Measurement and Quality Control

Chromosome arm-specific telomere lengths were measured by telomere quantitative fluorescent in situ hybridization (TQ-FISH) in combination with karyotyping by DAPI-banding (equivalent to G-banding). The chromosome preparations were dropped onto clean microscopic slides and fixed in crayon fixative for one hour, dehydrated through an ethanol series (70%, 80%, 90% and 100%), and air dried. Fifteen microliters of hybridization mixture consisting of 0.3 μg/ml Cy3-labeled telomere-specific peptide nucleic acid (PNA) probe (Panagene Inc., Daejeon, Korea), 1 ml of cocktails of FITC-labeled centromeric PNA probes specific for chromosomes 2, 4, 8, 9, 13, 15, 18, 20 and 21 (Biomarkers, Rockville, Md.), 20 mg/ml of Cy3-labeled centromeric PNA probes specific for chromosome X, 50% formamide, 10 mM Tris-HCl, pH 7.5, 5% blocking reagent, and 1×Denhart's solution was applied to each slide. Slides were then placed in a Hybex microarray hybridization oven (SciGene, Sunnyvale, Calif.) where the DNA was denatured by incubating at 75° C. for five minutes, followed by hybridizing at 30° C. for three hours. After hybridization, the slides were sequentially washed; once in 1×SSC, once in 0.5×SSC, and once in 0.1×SSC; each wash was 10 min at 42° C. The slides were then mounted in anti-fade mounting medium containing 300 ng/ml 4′-6-diamidino-2-phenylindole (DAPI).

After TQ-FISH, cells were analyzed using an epifluorescence microscope equipped with a charge-coupled device (CCD) camera (Leica Microsystems, Bannockburn, Ill.). Images were captured with exposure times of 0.15, 0.25 and 0.05 second for Cy3, FITC and DAPI signals, respectively. Digitized images were analyzed using a specialized Isis FISH imaging software with a telomere module (MetaSystems Inc. Boston, Mass.). This software permits measurement of 92 telomere signals simultaneously after karyotyping. Chromosome identification was achieved by: (1) DAPI banding (equivalent to G-banding); (2) chromosome specific centromere probes. Arm-specific telomere length measurements were made by telomere fluorescent in situ hybridization. Telomere sequences were labeled by a Cy3 (red) telomere specific PNA probe. Chromosomes 2, 4, 8, 9, 13, 15, 18, 20 and 21 were identified by combination of DAPI banding and chromosome specific centromeric probe (green signals). Chromosome X was identified by a Cy3 (red) labeled centromere probe.

Telomere fluorescent intensity units (FIU) were recorded as an indirect measurement of telomere length. Between the homologous telomeres, one telomere was often shorter than the other and there are significant differences in lengths between homologous telomeres. This observation is consistent with previous reports indicating that arm-specific telomere lengths were highly variable between chromosome arms and between homologous arms (Gilson, 2007; Graakjaer, 2006). Thus, each pair of homologous telomeres was recorded separately as homologous short (S) and homologous long (L). To normalize the FISH hybridization variations between samples, relative telomere length (RTL) was used for the subsequent statistical analysis. For each study subject, 15-17 metaphase cells were analyzed to estimate the mean relative telomere length for the 92 telomeres.

The definition of telomere-related parameters were as following: (1) relative telomere length (RTL) was defined as the ratio of the arm-specific telomere FIU to the total telomere FIU of 92 telomeres from the same cell; (2) homologous telomere length difference (HTLD) was defined as the percent of (homologous long TL−homologous short TL) divided by (homologous long TL+homologous short TL); (3) coefficient of variation (CV) of TL among 92 telomeres in a typical human cell was used as the measurement of telomere length variation.

Several quality control steps were used in this assay. Laboratory personnel who were responsible for the blood culture and telomere assay were blinded to the case-control status of the subjects. All new lots of reagents were tested to ensure optimal hybridization. A control slide containing cells with known total telomere length was included in each batch of TQ-FISH to monitor the quality of the hybridization efficiency. In this study, the coefficient of variation (CV) of overall telomere length from 20 repeats of the control slide was 12.4% and the correlations of telomere lengths between repeated samples were 0.89.

d) Statistical Analysis

Student t-test was used to compare the means of the variables (telomere length, homologous telomere length variation or telomere length variation in somatic cells) that were normally distributed and Wilcoxon rank sum test was used for non-normally distributed variables. Chi-square tests were used to compare the distribution of categorical variables between cases and controls. Pearson correlation was used to examine the correlations between telomere lengths, and between telomere lengths and age.

The associations between telomere-related parameters and the risk of breast cancer were examined using unconditional logistic regression. Relative telomere lengths, homologous telomere length variation and overall telomere length variation were dichotomized as short/long or high/low using the 50^{th} percentile values in the controls as a cut point. Telomere lengths were also categorized according to the quartiles in control subjects. Odds ratios were adjusted for age, race, smoking status, alcohol use, education, family history of cancer in first degree biological relatives, menopausal status, physical activity in the teens. P-values were two-sided and considered statistically significant if P<0.01. All analyses were performed using SAS software, version 9 (SAS Institute Inc., Cary, N.C.).

2. Results

a) Characteristics of Study Population

Table 6 lists the characteristics of the study subjects. The mean age is 53.0 for cases and 53.2 for controls. There are no significant case-control differences in the distributions of race, menopausal status, tobacco smoking status, alcohol use, education levels, family history of cancer, levels of household income and HRT use. The mean body mass index (BMI) was similar between cases and controls. Controls were significantly more likely to be physically active in both the teens and in the past year compared with cases. The mean age at first live birth is older in controls than in cases (Table 6).

TABLE 6
Distribution of Characteristics and
Known Breast Cancer Risk Factors

| | Cases | Controls | |
|---|---|---|---|
| | N = 205 | N = 236 | p-value |
| Demographic factors† | | | |
| Age (y) | 53.0 ± 11.0 | 53.2 ± 10.0 | 0.853 |
| Race, N (%) | | | |
| White | 74.1 | 71.5 | |
| Black | 20.3 | 25.0 | |
| Other | 5.6 | 3.5 | 0.343 |
| Education ≧ college (%) | 40.9 | 43.4 | 0.610 |
| Household Income ≧ 100K (%) | 55.0 | 56.1 | 0.850 |
| Reproductive risk factors† | | | |
| Age at menarche (y) | 12.6 ± 1.5 | 12.5 ± 1.8 | 0.583 |
| Postmenopausal (%) | 55.3 | 57.9 | 0.586 |
| Number of live births | 1.53 ± 1.31 | 1.51 ± 1.29 | 0.852 |
| Age at first live birth (y) | 27.1 ± 6.3 | 28.8 ± 6.6 | 0.042 |
| Used HRT‡ (%) | 32.8 | 39.8 | 0.138 |
| Other risk factors† | | | |
| Had FHC (%) | 57.8 | 52.3 | 0.268 |
| Body mass index | 27.3 ± 6.5 | 27.3 ± 7.1 | 0.970 |
| Ever smoked cigarettes (%) | 37.6 | 46.2 | 0.072 |
| Ever drank Alcohol (%) | 88.7 | 92.0 | 0.246 |
| Exercised regularly at teens (%) | 66.5 | 80.9 | <0.001 |

†Unless otherwise specified, mean ± SD are presented.
‡HRT = hormonal replacement therapy.
Exercised regularly was defined as any weekly physical activity that would make the subject sweat or increase their heart rate and last >20 minutes.
Family history of cancer (FHC) was defined as any cancer cases among 1^{st} degree blood relatives.

b) Telomere Length Correlation Between Chromosome Arms

Whether telomere length was correlated between chromosomal arms in control subjects was evaluated and it was determined that lengths between homologous telomeres were significantly correlated, Pearson correlation coefficient (r) ranged from 0.39 to 0.66 for 46 pairs of homologous telomeres (mean=0.52), all p-values <0.0001 (significant after Bonferroni correction for multiple comparisons 0.05/46=0.0011). In contrast, telomere lengths between non-homologous telomeres were not correlated, Pearson correlation coefficient ranged from −0.19 to 0.21, none of the p-value reached statistical significance after adjustment for multiple comparisons. Correlation between arm-specific telomere length and patient's age in control subjects was also examined. Significant correlations were observed for chromosome 2p-S (r=−0.22, p=0.0007) and 15p-S (r=−0.24, p=0.0003).

c) Arm-Specific Telomere Length and Breast Cancer Risk

Initial case-control comparison of mean RTLs identified four telomeres (1p-S, Xp-S, 9p-S and 15p-S) showed significant case-control difference at p<0.01 and one telomere (Xp-S) showed significant case-control difference at p<0.001 level (significant after Bonferroni correction for multiple comparisons 0.05/46=0.0011) in pre-menopausal women. In post-menopausal women, one telomere (15p-S) showed significant case-control difference at p<0.01 and none of the 46 telomeres showed significant case-control difference at p<0.001 level (Table 7). It is important to note that none of the homologous long version of the telomeres showed significant case-control differences (Table 14).

Because telomere lengths between homologous telomeres are significantly correlated, the analyses were focused on homologous short version of the 46 telomeres. Using the 50^{th} percentile value in controls as a cut point, multivariate logistic regression analysis confirmed that short telomere lengths on Xp-S and 15p-S were significantly associated with an increased breast cancer risk in premenopausal women, adjusted odds ratio (OR)=2.5 (95% CI=1.31 to 4.78) and 2.6 (95% CI=1.32 to 4.97) respectively (Table 8). ORs were adjusted for age, race, education, household income, physical activity in teens, smoking status, alcohol use and family history of cancer. When the study subjects were categorized into four groups (by quartiles) according to the telomere length, a highly significant inverse dose-response relationship was observed for Xp-S (P_{trend}=0.001) and 15p-S (P_{trend}=0.004), with the lowest-vs-highest quartile OR of 5.5 (95% CI=2.0 to 15.1) and 3.6 (95% CI=1.4 to 9.8) respectively (Table 8). In post-menopausal women, multivariate logistic regression analysis revealed that short telomere length on 15p-S was borderline significantly associated with an decreased breast cancer risk, adjusted OR=0.54 (95% CI=0.31 to 0.94). A significant dose-response relationship was also observed for 15p-S (P_{trend}=0.004, Table 8).

d) Telomere Length Variation Between Homologous Telomeres and Breast Cancer Risk

To test the hypothesis that increased telomere length variations across the genome will increase genomic instability, hence increased risk of breast cancer, whether differences in length between homologous telomeres are associated with breast cancer risk were examined. Homologous telomere length difference (HTLD) was defined as the percent of (homologous long TL−homologous short TL) divided by (homologous long TL+homologous short TL). Initial case-control comparison of mean HTLD identified seven chromosome arms (5q, Xp, 8q, 9p, 12p, 15p and 15q) showed significant case-control difference at p-value <0.01 level and three chromosome arms (5q, 9p and 15p) showed significant case-control difference at p-value <0.001 level (significant after Bonferroni correction for multiple comparisons 0.05/46=0.0011) in pre-menopausal women (Table 9). None of the 46 chromosome arms showed significant case-control difference in post-menopausal women.

Using the 50^{th} percentile value in controls as a cut point, multivariate logistic regression analysis confirmed that greater difference in length between homologous telomeres on chromosome arms 9p, 15p and 15q were significantly associated with an increased breast cancer risk in premenopausal women, adjusted odds ratio (OR)=4.6 (95% CI=2.3 to 9.2), 3.1 (1.6 to 6.0) and 2.8 (1.4 to 5.4) respectively (Table 10). When the study subjects were categorized into four groups (by quartiles) according to the telomere length, a significant dose-response relationship was observed for chromosome Xp (P_{trend}=0.005), 9p (P_{trend}<0.001), 15p (P_{trend}<0.001) and 15q (P_{trend}=0.005), respectively (Table 10). None of the chromosome arms showed significant association with breast cancer risk in post-menopausal women (Table 15).

e) Chromosome Arm-Specific Telomere Length Variation in Lymphocytes and Breast Cancer Risk

Telomere length variations among somatic cells (lymphocytes) were examined for their association with breast cancer risk. Fifteen to seventeen cells were assayed for each subject and standard deviations (SD) were computed for the RTL of each chromosome arm. The coefficient variation (CV) was used, which is the adjusted SD (CV=SD/mean), as the measurement of telomere length variation because the value of SD is related to the mean RTL and mean RTL is associated with breast cancer risk. The average CV of 46 chromosome arms (homologous telomeres were combined) was found to be significantly higher in cases (mean CV=43.7%) than in controls (mean CV=41.9%, p=1.50×10⁻⁷) in pre-menopausal women (Table 11). The same level significant case-control differences in mean CV were also observed for homologous short version of the 46 telomeres (p=6.48×10⁻⁷) and homologous long version of the 46 telomeres (p=6.77×10⁻⁸) in pre-menopausal women. We did not observe any significant case-control difference in average CV of 46 chromosome arms in post-menopausal women (Table 11).

Case-control comparison of mean CV of each chromosome arm identified seven chromosome arms (lp-L, 5q-S, 12p-L, 15p-S, 18p-S, 18p-L and 19q-L) showed significant case-control difference at p<0.01 level and none of the mean CV of the 92 chromosome arms showed significant case-control difference at p<0.0005 level (Bonferroni correction 0.05/92=0.0005) in pre-menopausal women (Table 12). In post-menopausal women, two chromosome arms (21p-S and 21p-L) showed significant case-control difference at p<0.01 level and none of the 92 chromosome arms showed significant case-control difference at p<0.0005 level (Table 12). Using the 50^{th} percentile value in controls as a cut point, multivariate logistic regression analysis revealed suggestive associations between the greater telomere length variations on chromosome arms 1p-L, 18p-S and 19q-L and an increased breast cancer risk in premenopausal women, adjusted OR=2.6 (95% CI=1.3 to 5.0), 2.4 (1.3 to 4.6), and 2.5 (1.3 to 4.9) respectively (Table 13). When the study subjects were categorized into four groups (by quartiles) according to the telomere length, a significant dose-response relationship was observed for chromosome 18p-S (P_{trend}=0.003) (Table 13). In post-menopausal women, greater telomere length variations on chromosome arms 15p-S and 21p-L were associated with a decreased breast cancer risk, adjusted OR=0.47 (95% CI=0.27 to 0.82) and 0.44 (0.25 to 0.77) respectively (Table 13). When the study subjects were categorized into four groups (by quartiles) according to the telomere length, a significant inverse dose-response relationship was observed for chromosome 15p-S (P_{trend}=0.006), and 21p-L (P_{trend} p=0.005) (Table 13).

TABLE 7
Case-Control Comparison of Mean Relative Telomere Length
(RTL) on homologous short version of chromosome arms

| | All subjects | | | Pre-menopausal women | | | Post-menopausal women | | |
|---|---|---|---|---|---|---|---|---|---|
| Chromosome | cases | controls | | cases | controls | | cases | controls | |
| arms | N = 204 | N = 236 | p-value† | N = 89 | N = 96 | p-value† | N = 110 | N = 132 | p-value† |
| 1p | 0.749 | 0.777 | 0.0432 | 0.744 | 0.805 | 0.0027 | 0.751 | 0.759 | 0.6687 |
| Xp | 0.783 | 0.802 | 0.2193 | 0.759 | 0.834 | 0.0008 | 0.780 | 0.778 | 0.2852 |
| 9p | 0.663 | 0.692 | 0.0239 | 0.665 | 0.719 | 0.0069 | 0.660 | 0.673 | 0.4449 |
| 15p | 0.700 | 0.697 | 0.7968 | 0.682 | 0.740 | 0.0073 | 0.713 | 0.664 | 0.0078 |

TABLE 8
Logistic Regression Examining the Association between Homologous Short RTL and Breast Cancer Risk

| | All subjects N = 440 | | Pre-menopausal women N = 185 | | Post-menopausal women N = 242 | |
|---|---|---|---|---|---|---|
| Chromosome arm | OR (95% CI) | p | OR (95% CI) | p | OR (95% CI) | p |
| 1p | | | | | | |
| By median | 1.35 (0.89-2.03) | 0.1549 | 1.73 (0.90-3.31) | 0.0994 | 1.08 (0.62-1.87) | 0.7869 |
| By quartiles | | | | | | |
| Q3 | 1.14 (0.63-2.05) | | 1.97 (0.80-4.84) | | 0.75 (0.33-1.71) | |
| Q2 | 1.15 (0.64-2.08) | | 1.96 (0.76-4.95) | | 0.72 (0.32-1.60) | |
| Q1 | 1.76 (0.99-3.13) | 0.0597* | 3.07 (1.20-7.86) | 0.0264* | 1.15 (0.54-2.45) | 0.6494* |
| Xp | | | | | | |
| By median | 1.12 (0.75-1.67) | 0.5920 | 2.50 (1.31-4.78) | 0.0055 | 0.59 (0.34-1.02) | 0.0603 |
| By quartiles | | | | | | |
| Q3 | 1.61 (0.90-2.86) | | 3.38 (1.31-8.69) | | 0.90 (0.40-2.02) | |
| Q2 | 1.17 (0.64-2.12) | | 5.21 (1.84-14.78) | | 0.37 (0.16-0.85) | |
| Q1 | 1.64 (0.92-2.91) | 0.2154* | 5.45 (1.97-15.05) | 0.0010* | 0.69 (0.32-1.49) | 0.1528* |
| 9p | | | | | | |
| By median | 1.13 (0.75-1.69) | 0.5632 | 1.42 (0.75-2.69) | 0.2764 | 1.02 (0.59-1.78) | 0.9366 |
| By quartiles | | | | | | |
| Q3 | 2.18 (1.21-3.91) | | 3.14 (1.28-7.72) | | 1.55 (0.68-3.54) | |
| Q2 | 1.55 (0.84-2.84) | | 2.23 (0.89-5.61) | | 1.27 (0.54-2.98) | |
| Q1 | 1.88 (1.04-3.41) | 0.1400* | 2.54 (1.00-6.43) | 0.0860* | 1.37 (0.61-3.08) | 0.6470* |
| 15p | | | | | | |
| By median | 1.05 (0.70-1.57) | 0.8318 | 2.56 (1.32-4.97) | 0.0054 | 0.54 (0.31-0.94) | 0.0283 |
| By quartiles | | | | | | |
| Q3 | 0.75 (0.42-1.33) | | 1.64 (0.67-4.03) | | 0.38 (0.17-0.87) | |
| Q2 | 1.02 (0.58-1.77) | | 2.99 (1.23-7.26) | | 0.41 (0.18-0.91) | |
| Q1 | 0.82 (0.46-1.44) | 0.7203* | 3.63 (1.35-9.75) | 0.0035* | 0.30 (0.14-0.64) | 0.0039* |

*p-for-trend
Bold p-values are significant at <0.01 level.
ORs were adjusted for age, race, education, household income, physical activity in teens, smoking status, alcohol use, family history of cancer and history of pregnancy.

TABLE 9
Case-Control Comparison of Mean Homologous Telomere Length Differences (HTLD)

| | All subjects | | | Pre-menopausal women | | | Post-menopausal women | | |
|---|---|---|---|---|---|---|---|---|---|
| Chromosome | cases | controls | | cases | controls | | cases | controls | |
| arms | N = 204 | N = 236 | p-value† | N = 89 | N = 96 | p-value† | N = 110 | N = 132 | p-value† |
| 5q | 38.45 | 36.21 | 0.0013 | 39.39 | 35.60 | 0.0006 | 37.93 | 36.61 | 0.1509 |
| Xp | 38.27 | 37.16 | 0.1280 | 39.37 | 36.40 | 0.0076 | 37.62 | 37.95 | 0.7445 |
| 8q | 40.68 | 38.48 | 0.0036 | 40.93 | 38.00 | 0.0077 | 40.80 | 38.90 | 0.0771 |
| 9p | 38.91 | 37.14 | 0.0169* | 39.10 | 35.90 | 0.0005* | 38.95 | 38.11 | 0.6494* |
| 12p | 38.80 | 36.86 | 0.0081 | 39.76 | 36.55 | 0.0058 | 38.18 | 37.30 | 0.3627 |
| 15p | 38.95 | 38.19 | 0.3038 | 40.14 | 35.87 | 0.0001 | 38.08 | 40.00 | 0.0533 |
| 15q | 38.13 | 36.55 | 0.0295 | 38.16 | 34.87 | 0.0034 | 38.20 | 37.99 | 0.8240 |

HTLD was defined as the percent of (homologous long T − homologous short T) divided by (homologous long T + homologous short T).
†all p-values were from t-test except for 9p.
*Wilcoxon rank sum test was used.
Bold p-values are significant after adjustment for multiple comparison (Bonferroni correction 0.05/46 = 0.0011).

TABLE 10
Logistic Regression Examining the Association between Allelic
Telomere Length Differences and Breast Cancer Risk

| | All subjects N = 440 | | Pre-menopausal women N = 185 | | Post-menopausal women N = 242 | |
|---|---|---|---|---|---|---|
| Chromosome arm | OR (95% CI) | p | OR (95% CI) | p | OR (95% CI) | p |
| 5q | | | | | | |
| By median | 1.56 (1.03-2.36) | 0.0356 | 1.92 (1.00-3.71) | 0.0508 | 1.47 (0.84-2.58) | 0.1817 |
| By quartiles | | | | | | |
| Q2 | 1.10 (0.63-1.89) | | 1.21 (0.51-2.83) | | 0.97 (0.46-2.04) | |
| Q3 | 1.49 (0.84-2.64) | | 1.41 (0.60-3.34) | | 1.75 (0.79-3.91) | |
| Q4 | 1.87 (1.05-3.32) | 0.0218 | 3.43 (1.32-8.88) | 0.0169 | 1.31 (0.61-2.85) | 0.2760 |
| Xp | | | | | | |
| By median | 1.31 (0.87-1.97) | 0.1904 | 1.82 (0.95-3.49) | 0.0727 | 1.08 (0.63-1.88) | 0.7719 |
| By quartiles | | | | | | |
| Q2 | 1.24 (0.71-2.18) | | 2.94 (1.15-7.55) | | 0.64 (0.30-1.38) | |
| Q3 | 1.24 (0.71-2.17) | | 2.16 (0.87-5.37) | | 0.84 (0.39-1.81) | |
| Q4 | 1.81 (1.02-3.21) | 0.0561 | 3.98 (1.63-9.70) | 0.0048 | 0.89 (0.40-2.00) | 0.9413 |
| 8q | | | | | | |
| By median | 1.37 (0.91-2.06) | 0.1283 | 1.63 (0.87-3.05) | 0.1311 | 1.26 (0.73-2.19) | 0.4089 |
| By quartiles | | | | | | |
| Q2 | 2.53 (1.41-4.54) | | 4.34 (1.72-10.92) | | 1.70 (0.77-3.80) | |
| Q3 | 1.65 (0.95-2.88) | | 3.03 (1.19-7.75) | | 1.17 (0.57-2.40) | |
| Q4 | 2.37 (1.35-4.16) | 0.0077 | 3.57 (1.44-8.84) | 0.0147 | 2.00 (0.94-4.24) | 0.1330 |
| 9p | | | | | | |
| By median | 1.92 (1.26-2.92) | 0.0022 | 4.59 (2.29-9.20) | <0.0001 | 1.07 (0.61-1.88) | 0.8262 |
| By quartiles | | | | | | |
| Q2 | 0.94 (0.55-1.62) | | 1.22 (0.46-3.21) | | 1.03 (0.50-2.12) | |
| Q3 | 1.82 (1.01-3.28) | | 7.18 (2.48-20.79) | | 0.83 (0.38-1.80) | |
| Q4 | 1.98 (1.09-3.58) | 0.0052 | 4.29 (1.53-11.99) | 0.0002 | 1.45 (0.65-3.16) | 0.5411 |
| 12p | | | | | | |
| By median | 1.25 (0.83-1.87) | 0.2863 | 2.01 (1.06-3.84) | 0.0334 | 0.86 (0.50-1.49) | 0.5944 |
| By quartiles | | | | | | |
| Q2 | 1.21 (0.70-2.11) | | 1.31 (0.54-3.18) | | 1.10 (0.51-2.36) | |
| Q3 | 1.28 (0.73-2.24) | | 2.29 (0.91-5.74) | | 0.82 (0.38-1.75) | |
| Q4 | 1.46 (0.83-2.58) | 0.1907 | 2.25 (0.94-5.40) | 0.0366 | 1.00 (0.46-2.20) | 0.8116 |
| 15p | | | | | | |
| By median | 1.18 (0.78-1.78) | 0.4311 | 3.06 (1.58-5.95) | 0.0010 | 0.58 (0.33-1.01) | 0.0536 |
| By quartiles | | | | | | |
| Q2 | 0.78 (0.45-1.37) | | 1.32 (0.50-3.45) | | 0.64 (0.30-1.36) | |
| Q3 | 0.91 (0.51-1.63) | | 2.56 (0.99-6.64) | | 0.48 (0.22-1.04) | |
| Q4 | 1.12 (0.63-1.99) | 0.6238 | 4.44 (1.70-11.60) | 0.0008 | 0.42 (0.19-0.95) | 0.0234 |
| 15q | | | | | | |
| By median | 1.58 (1.05-2.38) | 0.0295 | 2.79 (1.44-5.40) | 0.0023 | 1.16 (0.66-2.03) | 0.6176 |
| By quartiles | | | | | | |
| Q2 | 1.07 (0.62-1.84) | | 1.09 (0.42-2.83) | | 0.90 (0.44-1.80) | |
| Q3 | 1.62 (0.91-2.88) | | 2.91 (1.17-7.27) | | 1.12 (0.51-2.48) | |
| Q4 | 1.65 (0.92-2.95) | 0.0418 | 2.89 (1.17-7.16) | 0.0053 | 1.05 (0.46-2.38) | 0.7789 |

*p-for-trend.
Bold p-values are significant at <0.01 level.
ORs were adjusted for age, race, education, household income, physical activity in teens, smoking status, alcohol use, family history of cancer and history of pregnancy.

TABLE 11
Comparing the Average Coefficient Variation (CV) of 46 Telomeres Between Cases and Controls

| | All subjects | | | Pre-menopausal women | | | Post-menopausal women | | |
|---|---|---|---|---|---|---|---|---|---|
| | cases | controls | | cases | controls | | cases | controls | |
| Type of telomeres | N = 204 | N = 236 | p-value† | N = 89 | N = 96 | p-value† | N = 110 | N = 132 | p-value† |
| Short version (S) | 64.52 | 63.16 | 0.0020 | 64.90 | 62.21 | 6.48 × 10⁻⁷ | 64.49 | 64.17 | 0.4537 |
| Long Version (L) | 44.22 | 43.36 | 0.0015 | 44.46 | 42.59 | 6.77 × 10⁻⁸ | 44.14 | 44.08 | 0.8903 |
| Combined (S + L) | 43.39 | 42.60 | 0.0025 | 43.67 | 41.90 | 1.50 × 10⁻⁷ | 43.30 | 43.29 | 0.9695 |

CV is expressed as %.
†p-values were from t-test.

TABLE 12
Case-Control comparison of Mean Coefficient of Variation (CV) of Telomere Length

| | All subjects | | | Pre-menopausal women | | | Post-menopausal women | | |
|---|---|---|---|---|---|---|---|---|---|
| Chromosome | cases | controls | | cases | controls | | cases | controls | |
| arms | N = 204 | N = 236 | p-value† | N = 89 | N = 96 | p-value† | N = 110 | N = 132 | p-value† |
| 1p-AL* | 43.78 | 42.42 | 0.1076 | 45.05 | 41.23 | 0.0054 | 42.88 | 43.36 | 0.6780 |
| 5q-AS\hat{ } | 65.14 | 61.34 | 0.0073 | 66.14 | 59.95 | 0.0053 | 64.56 | 62.31 | 0.2271 |
| 8q-AS | 70.90 | 66.25 | 0.0051 | 71.67 | 65.94 | 0.0423 | 70.84 | 66.86 | 0.0569 |
| 12p-AL | 44.91 | 42.76 | 0.0174 | 45.96 | 41.45 | 0.0037 | 44.07 | 43.59 | 0.5355 |
| 15p-AS | 65.11 | 64.77 | 0.8731 | 66.42 | 61.26 | 0.0085 | 64.07 | 67.73 | 0.0606 |
| 18p-AS | 60.87 | 59.28 | 0.2380 | 61.93 | 56.85 | 0.0048 | 60.37 | 61.42 | 0.5972 |
| 18p-AL | 43.81 | 43.32 | 0.1121 | 44.41 | 40.42 | 0.0049 | 43.40 | 43.86 | 0.7310 |
| 19q-AL | 44.76 | 44.12 | 0.5118 | 46.29 | 42.82 | 0.0097 | 43.66 | 45.24 | 0.2115 |
| 21p-AS | 65.26 | 67.43 | 0.1002 | 66.47 | 66.25 | 0.9606 | 64.04 | 69.02 | 0.0074 |
| 21p-AL | 45.88 | 46.71 | 0.3556 | 46.94 | 45.07 | 0.1425 | 44.75 | 48.26 | 0.0090 |

*AL = allelically long version.
\hat{ }AS = allelically short version.
†Wilcoxon rank sum test was used for 5q-AS, 8q-AS, 12p-AL and 21p-AS, t-test was used for the other 6 telomeres.
CV is expressed as %.

TABLE 13
Logistic Regression Examining the Association between
Telomere Length Variation (TLV) and Breast Cancer Risk

| | All subjects N = 440 | | Pre-menopausal women N = 185 | | Post-menopausal women N = 242 | |
|---|---|---|---|---|---|---|
| Chromosome arm | OR (95% CI) | p | OR (95% CI) | p | OR (95% CI) | p |
| 1p-AL | | | | | | |
| By median | 1.46 (0.97-2.19) | 0.0687 | 2.58 (1.33-5.02) | 0.0052 | 0.99 (0.57-1.71) | 0.9616 |
| By quartiles | | | | | | |
| Q2 | 0.72 (0.42-1.25) | | 0.74 (0.31-1.76) | | 0.62 (0.29-1.31) | |
| Q3 | 1.13 (0.63-2.03) | | 2.46 (0.94-6.47) | | 0.65 (0.30-1.43) | |
| Q4 | 1.33 (0.74-2.39) | 0.1624 | 1.99 (0.79-5.02) | 0.0313 | 0.90 (0.40-2.02) | 0.8275 |
| 5q-AS | | | | | | |
| By median | 1.26 (0.84-1.90) | 0.2634 | 1.78 (0.93-3.40) | 0.0798 | 1.01 (0.58-1.75) | 0.9800 |
| By quartiles | | | | | | |
| Q2 | 1.55 (0.89-2.71) | | 1.91 (0.75-4.87) | | 1.38 (0.66-2.89) | |
| Q3 | 1.22 (0.71-2.10) | | 1.71 (0.73-4.02) | | 0.95 (0.46-1.98) | |
| Q4 | 2.08 (1.15-3.76) | 0.0418 | 3.91 (1.47-10.42) | 0.0121 | 1.49 (0.67-3.31) | 0.5367 |
| 8q-AS | | | | | | |
| By median | 1.75 (1.15-2.65) | 0.0088 | 1.76 (0.93-3.32) | 0.0814 | 1.92 (1.07-3.43) | 0.0279 |
| By quartiles | | | | | | |
| Q2 | 0.76 (0.44-1.28) | | 1.15 (0.49-2.71) | | 0.52 (0.25-1.06) | |
| Q3 | 1.74 (0.93-3.28) | | 2.44 (0.93-6.41) | | 1.49 (0.62-3.56) | |
| Q4 | 1.32 (0.74-2.37) | 0.0824 | 1.54 (0.62-3.86) | 0.1707 | 1.25 (0.56-2.80) | 0.2192 |
| 12p-AL | | | | | | |
| By median | 1.56 (1.04-2.34) | 0.0331 | 2.34 (1.21-4.53) | 0.0115 | 1.17 (0.68-2.02) | 0.5803 |
| By quartiles | | | | | | |
| Q2 | 0.97 (0.56-1.68) | | 0.65 (0.26-1.63) | | 1.10 (0.52-2.32) | |
| Q3 | 1.44 (0.82-2.52) | | 2.12 (0.87-5.19) | | 1.09 (0.50-2.37) | |
| Q4 | 1.65 (0.92-2.95) | 0.0461 | 1.85 (0.75-4.55) | 0.0499 | 1.40 (0.62-3.15) | 0.4554 |
| 15p-AS | | | | | | |
| By median | 0.78 (0.52-1.17) | 0.2359 | 1.57 (0.83-2.98) | 0.1696 | 0.47 (0.27-0.82) | 0.0085 |
| By quartiles | | | | | | |
| Q2 | 0.94 (0.52-1.69) | | 1.50 (0.58-3.92) | | 0.67 (0.31-1.53) | |
| Q3 | 0.69 (0.39-1.20) | | 1.41 (0.58-3.44) | | 0.43 (0.19-0.94) | |
| Q4 | 0.85 (0.48-1.51) | 0.3807 | 3.10 (1.15-8.34) | 0.0389 | 0.37 (0.17-0.80) | 0.0061 |
| 18p-AS | | | | | | |
| By median | 1.33 (0.88-1.99) | 0.1725 | 2.40 (1.26-4.60) | 0.0080 | 0.83 (0.48-1.44) | 0.4985 |
| By quartiles | | | | | | |
| Q2 | 1.13 (0.64-1.97) | | 1.58 (0.65-3.86) | | 1.00 (0.47-2.15) | |
| Q3 | 1.18 (0.68-2.05) | | 2.42 (0.94-6.20) | | 0.72 (0.35-1.51) | |
| Q4 | 1.80 (1.00-3.25) | 0.0642 | 4.27 (1.5-11.58) | 0.0026 | 1.03 (0.47-2.25) | 0.7911 |
| 18p-AL | | | | | | |
| By median | 0.99 (0.66-1.48) | 0.9516 | 1.70 (0.89-3.22) | 0.1066 | 0.66 (0.38-1.15) | 0.1394 |
| By quartiles | | | | | | |
| Q2 | 0.92 (0.52-1.63) | | 1.12 (0.44-2.87) | | 0.91 (0.42-1.95) | |
| Q3 | 0.77 (0.44-1.35) | | 1.22 (0.48-3.14) | | 0.56 (0.27-1.16) | |
| Q4 | 1.36 (0.75-2.47) | 0.5065 | 2.90 (1.08-7.83) | 0.0355 | 0.91 (0.41-2.04) | 0.4543 |
| 19q-AL | | | | | | |
| By median | 1.10 (0.73-1.65) | 0.6472 | 2.54 (1.32-4.89) | 0.0054 | 0.53 (0.30-0.93) | 0.0274 |
| By quartiles | | | | | | |
| Q2 | 0.79 (0.45-1.38) | | 0.87 (0.36-2.09) | | 0.74 (0.34-1.64) | |
| Q3 | 1.08 (0.60-1.94) | | 2.82 (1.04-7.64) | | 0.52 (0.23-1.16) | |
| Q4 | 0.89 (0.50-1.56) | 0.9500 | 2.08 (0.85-5.09) | 0.0248 | 0.40 (0.18-0.88) | 0.0161 |
| 21p-AS | | | | | | |
| By median | 0.75 (0.50-1.13) | 0.1642 | 1.07 (0.56-2.04) | 0.8349 | 0.54 (0.31-0.93) | 0.0268 |
| By quartiles | | | | | | |
| Q2 | 0.95 (0.53-1.71) | | 1.22 (0.49-3.02) | | 0.90 (0.40-2.01) | |
| Q3 | 0.76 (0.43-1.36) | | 1.16 (0.45-2.98) | | 0.57 (0.26-1.23) | |
| Q4 | 0.70 (0.40-1.25) | 0.1652 | 1.22 (0.49-3.03) | 0.7212 | 0.45 (0.20-0.98) | 0.0240 |
| 21p-AL | | | | | | |
| By median | 0.70 (0.46-1.05) | 0.0806 | 1.20 (0.64-2.27) | 0.5729 | 0.44 (0.25-0.77) | 0.0042 |
| By quartiles | | | | | | |
| Q2 | 1.13 (0.61-2.07) | | 1.45 (0.54-3.93) | | 1.10 (0.47-2.55) | |
| Q3 | 0.71 (0.40-1.26) | | 1.08 (0.43-2.75) | | 0.56 (0.26-1..22) | |
| Q4 | 0.77 (0.43-1.38) | 0.1815 | 2.22 (0.83-5.93) | 0.1905 | 0.40 (0.18-0.81) | 0.0045 |

*p-for-trend.
Bold p-values are significant at < 0.01 level.
ORs were adjusted for age, race, education, household income, physical activity in teens, smoking status, alcohol use, family history of cancer and history of pregnancy.
TLV is the coefficient variation (CV) of telomere length in 15 cells that were from the same individual.

TABLE 14
Case-Control Comparison of Mean Relative Telomere Length (RTL\hat{ })

| | All subjects | | | Pre-menopausal women | | | Post-menopausal women | | |
|---|---|---|---|---|---|---|---|---|---|
| Chromosome | cases | controls | | cases | controls | | cases | controls | |
| arms | N = 204 | N = 236 | p† | N = 89 | N = 96 | p† | N = 110 | N = 132 | p† |

Short version of homologous telomeres

| | | | | | | | | | |
|---|---|---|---|---|---|---|---|---|---|
| 1p | 0.749 | 0.777 | 0.0432 | 0.744 | 0.805 | 0.0027 | 0.751 | 0.759 | 0.6687 |
| 1q | 0.773 | 0.778 | 0.7450 | 0.761 | 0.787 | 0.2623 | 0.780 | 0.770 | 0.6174 |
| 2p | 0.819 | 0.812 | 0.6207 | 0.825 | 0.830 | 0.8251 | 0.819 | 0.796 | 0.2522 |
| 2q | 0.692 | 0.707 | 0.2188 | 0.696 | 0.711 | 0.3823 | 0.687 | 0.703 | 0.3842 |
| 3p | 0.861 | 0.878 | 0.2829 | 0.863 | 0.879 | 0.5319 | 0.860 | 0.879 | 0.3825 |
| 3q | 0.752 | 0.752 | 0.9970 | 0.745 | 0.749 | 0.8398 | 0.755 | 0.750 | 0.7727 |
| 4p | 0.757 | 0.768 | 0.3967 | 0.759 | 0.791 | 0.0943 | 0.758 | 0.750 | 0.6999 |
| 4q | 0.837 | 0.860 | 0.1430 | 0.828 | 0.853 | 0.2843 | 0.843 | 0.861 | 0.3880 |
| 5p | 0.828 | 0.810 | 0.2664 | 0.839 | 0.807 | 0.1633 | 0.818 | 0.805 | 0.5354 |
| 5q | 0.707 | 0.741 | 0.0109 | 0.701 | 0.738 | 0.0609 | 0.711 | 0.745 | 0.0723 |
| 6p | 0.711 | 0.714 | 0.8081 | 0.691 | 0.713 | 0.2983 | 0.721 | 0.709 | 0.4974 |
| 6q | 0.779 | 0.794 | 0.3215 | 0.798 | 0.804 | 0.8083 | 0.765 | 0.789 | 0.2076 |
| 7p | 0.702 | 0.705 | 0.8462 | 0.715 | 0.721 | 0.7689 | 0.692 | 0.693 | 0.9490 |
| 7q | 0.780 | 0.800 | 0.1918 | 0.786 | 0.789 | 0.8932 | 0.775 | 0.801 | 0.1778 |
| 8p | 0.782 | 0.776 | 0.6847 | 0.803 | 0.777 | 0.2536 | 0.763 | 0.777 | 0.4329 |
| 8q | 0.650 | 0.676 | 0.0414 | 0.660 | 0.678 | 0.3838 | 0.638 | 0.678 | 0.0263 |
| 9p | 0.663 | 0.692 | 0.0239 | 0.665 | 0.719 | 0.0069 | 0.660 | 0.673 | 0.4449 |
| 9q | 0.645 | 0.640 | 0.6731 | 0.643 | 0.632 | 0.4798 | 0.649 | 0.644 | 0.8103 |
| 10p | 0.769 | 0.762 | 0.6309 | 0.770 | 0.746 | 0.1987 | 0.758 | 0.774 | 0.3728 |
| 10q | 0.726 | 0.725 | 0.9586 | 0.713 | 0.721 | 0.7172 | 0.738 | 0.730 | 0.6724 |
| 11p | 0.712 | 0.707 | 0.7095 | 0.717 | 0.702 | 0.4761 | 0.705 | 0.704 | 0.9725 |
| 11q | 0.715 | 0.721 | 0.5972 | 0.712 | 0.701 | 0.5536 | 0.720 | 0.738 | 0.3159 |
| 12p | 0.638 | 0.656 | 0.1413 | 0.621 | 0.667 | 0.0146 | 0.653 | 0.648 | 0.7462 |
| 12q | 0.740 | 0.748 | 0.5735 | 0.736 | 0.768 | 0.0995 | 0.742 | 0.731 | 0.5719 |
| 13p | 0.720 | 0.706 | 0.3322 | 0.743 | 0.702 | 0.0633 | 0.705 | 0.710 | 0.8226 |
| 13q | 0.771 | 0.783 | 0.4168 | 0.767 | 0.790 | 0.3312 | 0.776 | 0.779 | 0.8589 |
| 14p | 0.721 | 0.711 | 0.4879 | 0.728 | 0.716 | 0.6015 | 0.708 | 0.709 | 0.9395 |
| 14q | 0.721 | 0.714 | 0.6078 | 0.720 | 0.729 | 0.6778 | 0.723 | 0.703 | 0.2811 |
| 15p | 0.700 | 0.697 | 0.7968 | 0.682 | 0.740 | 0.0073 | 0.713 | 0.664 | 0.0078 |
| 15q | 0.670 | 0.682 | 0.2992 | 0.670 | 0.706 | 0.0591 | 0.669 | 0.665 | 0.8421 |
| 16p | 0.626 | 0.631 | 0.6975 | 0.627 | 0.627 | 0.9869 | 0.622 | 0.631 | 0.5676 |
| 16q | 0.646 | 0.660 | 0.2198 | 0.634 | 0.673 | 0.0314 | 0.652 | 0.650 | 0.8772 |
| 17p | 0.590 | 0.603 | 0.2169 | 0.610 | 0.615 | 0.7768 | 0.576 | 0.596 | 0.1455 |
| 17q | 0.611 | 0.603 | 0.4947 | 0.608 | 0.604 | 0.7911 | 0.609 | 0.602 | 0.6577 |
| 18p | 0.718 | 0.726 | 0.5698 | 0.709 | 0.743 | 0.1142 | 0.724 | 0.715 | 0.6462 |
| 18q | 0.710 | 0.720 | 0.4812 | 0.689 | 0.718 | 0.1746 | 0.725 | 0.716 | 0.6219 |
| 19p | 0.583 | 0.587 | 0.7042 | 0.595 | 0.591 | 0.8143 | 0.570 | 0.581 | 0.4629 |
| 19q | 0.622 | 0.629 | 0.5405 | 0.606 | 0.625 | 0.3208 | 0.630 | 0.631 | 0.9635 |
| 20p | 0.628 | 0.635 | 0.5656 | 0.631 | 0.637 | 0.7191 | 0.625 | 0.633 | 0.5789 |
| 20q | 0.572 | 0.577 | 0.6136 | 0.573 | 0.570 | 0.8473 | 0.571 | 0.583 | 0.4073 |
| 21p | 0.678 | 0.653 | 0.0713 | 0.679 | 0.671 | 0.6857 | 0.679 | 0.636 | 0.0273 |
| 21q | 0.594 | 0.595 | 0.9477 | 0.594 | 0.595 | 0.9757 | 0.591 | 0.592 | 0.9214 |
| 22p | 0.675 | 0.686 | 0.4403 | 0.656 | 0.674 | 0.4151 | 0.693 | 0.686 | 0.6917 |
| 22q | 0.576 | 0.580 | 0.6673 | 0.564 | 0.583 | 0.2409 | 0.583 | 0.579 | 0.7821 |
| Xp | 0.783 | 0.802 | 0.2193 | 0.759 | 0.834 | 0.0008 | 0.780 | 0.778 | 0.2852 |
| Xq | 0.718 | 0.713 | 0.6992 | 0.718 | 0.728 | 0.6179 | 0.718 | 0.703 | 0.4206 |
| 1p | 1.558 | 1.594 | 0.1130 | 1.540 | 1.607 | 0.0476 | 1.574 | 1.595 | 0.5076 |
| 1q | 1.584 | 1.586 | 0.9305 | 1.570 | 1.583 | 0.6748 | 1.599 | 1.588 | 0.7027 |
| 2p | 1.652 | 1.656 | 0.8485 | 1.655 | 1.650 | 0.8636 | 1.657 | 1.663 | 0.8362 |
| 2q | 1.439 | 1.439 | 0.9889 | 1.467 | 1.422 | 0.1108 | 1.422 | 1.447 | 0.3470 |
| 3p | 1.751 | 1.754 | 0.9002 | 1.751 | 1.738 | 0.7133 | 1.750 | 1.768 | 0.5239 |
| 3q | 1.538 | 1.500 | 0.0397 | 1.553 | 1.486 | 0.0150 | 1.530 | 1.502 | 0.3006 |
| 4p | 1.545 | 1.559 | 0.4797 | 1.565 | 1.560 | 0.8812 | 1.524 | 1.557 | 0.1996 |
| 4q | 1.706 | 1.720 | 0.5342 | 1.682 | 1.695 | 0.6953 | 1.726 | 1.734 | 0.7906 |
| 5p | 1.712 | 1.690 | 0.3525 | 1.707 | 1.656 | 0.1565 | 1.717 | 1.711 | 0.8499 |
| 5q | 1.500 | 1.503 | 0.8568 | 1.512 | 1.465 | 0.1215 | 1.494 | 1.533 | 0.1666 |
| 6p | 1.486 | 1.478 | 0.6886 | 1.472 | 1.482 | 0.7660 | 1.491 | 1.472 | 0.4690 |
| 6q | 1.623 | 1.613 | 0.6289 | 1.647 | 1.603 | 0.1757 | 1.609 | 1.617 | 0.7564 |
| 7p | 1.486 | 1.492 | 0.7771 | 1.497 | 1.480 | 0.6127 | 1.483 | 1.506 | 0.3785 |
| 7q | 1.626 | 1.630 | 0.8233 | 1.646 | 1.590 | 0.0860 | 1.609 | 1.661 | 0.0621 |
| 8p | 1.625 | 1.596 | 0.2010 | 1.620 | 1.604 | 0.6214 | 1.628 | 1.597 | 0.3064 |
| 8q | 1.454 | 1.444 | 0.6300 | 1.480 | 1.433 | 0.1381 | 1.434 | 1.459 | 0.3865 |
| 9p | 1.438 | 1.427 | 0.5605 | 1.450 | 1.455 | 0.8674 | 1.432 | 1.411 | 0.4235 |
| 9q | 1.343 | 1.338 | 0.7943 | 1.323 | 1.311 | 0.6818 | 1.357 | 1.349 | 0.7370 |
| 10p | 1.567 | 1.561 | 0.7671 | 1.576 | 1.536 | 0.1814 | 1.551 | 1.581 | 0.2758 |
| 10q | 1.523 | 1.507 | 0.4516 | 1.519 | 1.486 | 0.2821 | 1.529 | 1.521 | 0.7815 |
| 11p | 1.468 | 1.476 | 0.7278 | 1.499 | 1.484 | 0.6518 | 1.445 | 1.465 | 0.4936 |
| 11q | 1.479 | 1.463 | 0.4166 | 1.469 | 1.439 | 0.2736 | 1.494 | 1.489 | 0.8556 |
| 12p | 1.368 | 1.338 | 0.0770 | 1.364 | 1.348 | 0.5541 | 1.376 | 1.335 | 0.0723 |
| 12q | 1.599 | 1.595 | 0.8535 | 1.623 | 1.602 | 0.5009 | 1.581 | 1.585 | 0.8941 |
| 13p | 1.522 | 1.517 | 0.8026 | 1.507 | 1.519 | 0.6806 | 1.541 | 1.523 | 0.5660 |
| 13q | 1.619 | 1.602 | 0.4375 | 1.620 | 1.591 | 0.3770 | 1.621 | 1.609 | 0.7071 |
| 14p | 1.542 | 1.528 | 0.5462 | 1.556 | 1.525 | 0.4115 | 1.526 | 1.534 | 0.7942 |
| 14q | 1.498 | 1.502 | 0.8396 | 1.490 | 1.483 | 0.8021 | 1.508 | 1.520 | 0.6663 |
| 15p | 1.525 | 1.489 | 0.1193 | 1.521 | 1.497 | 0.4909 | 1.529 | 1.485 | 0.1700 |
| 15q | 1.428 | 1.392 | 0.0437 | 1.432 | 1.395 | 0.1633 | 1.427 | 1.394 | 0.1987 |
| 16p | 1.323 | 1.347 | 0.2433 | 1.321 | 1.329 | 0.7978 | 1.322 | 1.356 | 0.2451 |
| 16q | 1.371 | 1.374 | 0.8659 | 1.355 | 1.370 | 0.6228 | 1.381 | 1.370 | 0.6608 |
| 17p | 1.278 | 1.264 | 0.3967 | 1.304 | 1.262 | 0.1210 | 1.264 | 1.270 | 0.7949 |
| 17q | 1.287 | 1.281 | 0.7476 | 1.284 | 1.289 | 0.8712 | 1.288 | 1.275 | 0.5968 |
| 18p | 1.432 | 1.427 | 0.7862 | 1.415 | 1.453 | 0.1838 | 1.444 | 1.412 | 0.2018 |
| 18q | 1.491 | 1.474 | 0.4166 | 1.476 | 1.458 | 0.6247 | 1.503 | 1.482 | 0.4723 |
| 19p | 1.247 | 1.245 | 0.9397 | 1.268 | 1.265 | 0.9183 | 1.233 | 1.230 | 0.9170 |
| 19q | 1.329 | 1.301 | 0.1137 | 1.319 | 1.295 | 0.4053 | 1.337 | 1.308 | 0.1844 |
| 20p | 1.355 | 1.348 | 0.6971 | 1.352 | 1.349 | 0.9081 | 1.354 | 1.345 | 0.7389 |
| 20q | 1.230 | 1.211 | 0.2732 | 1.246 | 1.198 | 0.0665 | 1.220 | 1.226 | 0.7742 |
| 21p | 1.472 | 1.415 | 0.0106 | 1.467 | 1.406 | 0.0723 | 1.483 | 1.421 | 0.0466 |
| 21q | 1.264 | 1.245 | 0.2524 | 1.263 | 1.244 | 0.4407 | 1.264 | 1.247 | 0.4719 |
| 22p | 1.454 | 1.474 | 0.3597 | 1.461 | 1.468 | 0.8557 | 1.455 | 1.473 | 0.5368 |
| 22q | 1.229 | 1.237 | 0.6577 | 1.211 | 1.229 | 0.4840 | 1.242 | 1.247 | 0.8456 |
| Xp | 1.675 | 1.667 | 0.7568 | 1.657 | 1.693 | 0.3217 | 1.692 | 1.656 | 0.2745 |
| Xq | 1.471 | 1.462 | 0.6379 | 1.483 | 1.454 | 0.2846 | 1.465 | 1.474 | 0.7373 |

\hat{ }RTL was defined as the percent of arm-specific telomere fluorescent intensity unites (FIU) divided by total telomere FIU of 92 telomeres.
†all p-values were based on Student t-test.
Bold p-values were significant at <0.01 level.

TABLE 15
Case-Control Comparison of Mean Homologous Telomere Length Differences (HTLD)

| | All subjects | | | Pre-menopausal women | | | Post-menopausal women | | |
|---|---|---|---|---|---|---|---|---|---|
| Chromosome | cases | controls | | cases | controls | | cases | controls | |
| arms | N = 204 | N = 236 | p† | N = 89 | N = 96 | p† | N = 110 | N = 132 | p† |
| 1p | 36.76 | 36.28 | 0.4977 | 36.43 | 34.83 | 0.1483 | 37.30 | 37.50 | 0.8336 |
| 1q | 36.81 | 36.36 | 0.5423 | 37.21 | 35.50 | 0.1215 | 36.85 | 37.14 | 0.7852 |
| 2p | 36.24 | 36.40 | 0.8255 | 35.86 | 34.92 | 0.3768 | 36.52 | 37.68 | 0.2336 |
| 2q | 37.06 | 36.28 | 0.2428 | 37.32 | 35.59 | 0.0790 | 37.24 | 36.86 | 0.6821 |
| 3p | 36.21 | 35.33 | 0.2051 | 36.13 | 35.30 | 0.4352 | 36.29 | 35.40 | 0.3446 |
| 3q | 36.21 | 35.18 | 0.1223 | 36.88 | 34.89 | 0.0553 | 35.87 | 35.43 | 0.6310 |
| 4p | 36.51 | 36.10 | 0.5403 | 37.06 | 34.83 | 0.0231 | 35.88 | 37.17 | 0.1656 |
| 4q | 36.28 | 35.02 | 0.0782 | 36.29 | 34.56 | 0.1134 | 36.38 | 35.39 | 0.3174 |
| 5p | 36.88 | 37.03 | 0.8358 | 36.21 | 36.33 | 0.9109 | 37.46 | 37.76 | 0.7680 |
| 5q | 38.45 | 36.21 | 0.0013 | 39.39 | 35.60 | 0.0006 | 37.93 | 36.61 | 0.1509 |
| 6p | 37.77 | 36.82 | 0.2181 | 38.73 | 36.81 | 0.1214 | 37.12 | 37.06 | 0.9517 |
| 6q | 37.48 | 36.19 | 0.0908 | 36.80 | 35.18 | 0.1674 | 38.13 | 36.76 | 0.1803 |
| 7p | 37.63 | 37.55 | 0.9123 | 37.04 | 36.36 | 0.5393 | 38.32 | 38.53 | 0.8267 |
| 7q | 37.33 | 36.38 | 0.2079 | 37.66 | 35.82 | 0.1018 | 37.09 | 37.10 | 0.9896 |
| 8p | 37.06 | 36.78 | 0.6739 | 35.84 | 36.96 | 0.2828 | 38.11 | 36.82 | 0.1420 |
| 8q | 40.68 | 38.48 | 0.0036 | 40.93 | 38.00 | 0.0077 | 40.80 | 38.90 | 0.0771 |
| 9p* | 38.91 | 37.14 | 0.0169 | 39.10 | 35.90 | 0.0005 | 38.95 | 38.11 | 0.6494 |
| 9q | 36.89 | 37.22 | 0.6576 | 6.34 | 36.99 | 0.543 | 37.22 | 3734 | 0.9118 |
| 10p | 36.22 | 36.39 | 0.8100 | 36.35 | 36.62 | 0.7965 | 36.46 | 36.39 | 0.9461 |
| 10q | 37.39 | 37.25 | 0.8512 | 37.91 | 36.93 | 0.4078 | 36.94 | 37.43 | 0.6193 |
| 11p | 36.69 | 36.98 | 0.6725 | 37.59 | 37.48 | 0.9130 | 36.21 | 36.89 | 0.4821 |
| 11q | 36.95 | 36.36 | 0.3937 | 36.96 | 36.81 | 0.8871 | 37.00 | 36.25 | 0.4278 |
| 12p | 38.80 | 36.86 | 0.0081 | 39.76 | 36.55 | 0.0058 | 38.18 | 37.30 | 0.3627 |
| 12q | 38.80 | 38.12 | 0.3464 | 39.59 | 37.44 | 0.0425 | 38.44 | 38.70 | 0.7988 |
| 13p | 38.01 | 38.51 | 0.4929 | 36.17 | 38.99 | 0.0124 | 39.51 | 38.40 | 0.2659 |
| 13q | 37.61 | 36.45 | 0.1077 | 38.21 | 35.86 | 0.0480 | 37.19 | 36.90 | 0.7532 |
| 14p | 38.63 | 38.56 | 0.9202 | 38.89 | 38.20 | 0.5333 | 38.72 | 38.90 | 0.8550 |
| 14q | 37.31 | 37.69 | 0.5887 | 37.38 | 36.20 | 0.2735 | 37.31 | 38.87 | 0.1029 |
| 15p | 38.95 | 38.19 | 0.3038 | 40.14 | 35.87 | 0.0001 | 38.08 | 40.00 | 0.0533 |
| 15q | 38.13 | 36.55 | 0.0295 | 38.16 | 34.87 | 0.0034 | 38.20 | 37.99 | 0.8240 |
| 16p | 38.17 | 37.98 | 0.7831 | 38.12 | 37.95 | 0.8701 | 38.41 | 38.07 | 0.7142 |
| 16q | 37.68 | 36.93 | 0.2885 | 37.94 | 36.31 | 0.1357 | 37.59 | 37.30 | 0.7586 |
| 17p | 39.63 | 37.71 | 0.0038 | 39.27 | 37.49 | 0.0866 | 39.82 | 37.90 | 0.0346 |
| 17q | 38.01 | 38.19 | 0.8107 | 38.15 | 38.62 | 0.6774 | 38.16 | 37.84 | 0.7460 |
| 18p | 34.98 | 34.63 | 0.6228 | 35.17 | 34.06 | 0.2923 | 34.90 | 35.18 | 0.7841 |
| 18q | 37.83 | 36.54 | 0.0789 | 38.79 | 36.24 | 0.0249 | 37.28 | 37.05 | 0.8172 |
| 19p | 38.16 | 37.86 | 0.6610 | 38.06 | 38.08 | 0.9824 | 38.55 | 37.83 | 0.4400 |
| 19q | 38.71 | 37.07 | 0.0308 | 39.44 | 37.31 | 0.0557 | 38.43 | 37.06 | 0.1963 |
| 20p | 38.87 | 38.01 | 0.2295 | 38.42 | 37.62 | 0.4612 | 39.12 | 38.27 | 0.3467 |
| 20q | 38.61 | 37.30 | 0.0648 | 39.30 | 36.96 | 0.0362 | 38.05 | 37.67 | 0.6911 |
| 21p | 38.68 | 38.87 | 0.7816 | 38.37 | 37.46 | 0.3888 | 38.95 | 40.22 | 0.1951 |
| 21q | 38.32 | 37.52 | 0.2763 | 38.03 | 37.46 | 0.5981 | 38.72 | 37.75 | 0.3421 |
| 22p | 39.04 | 38.96 | 0.9072 | 40.50 | 39.19 | 0.2606 | 37.95 | 39.16 | 0.2106 |
| 22q | 38.44 | 38.22 | 0.7650 | 38.76 | 37.58 | 0.2785 | 38.32 | 38.86 | 0.5914 |
| Xp | 38.27 | 37.16 | 0.1280 | 39.37 | 36.40 | 0.0076 | 37.62 | 37.95 | 0.7445 |
| Xq | 36.65 | 36.22 | 0.5363 | 36.88 | 35.42 | 0.1641 | 36.62 | 37.04 | 0.6720 |

HTLD was defined as the percent of (homologous long RTL − homologous short RTL) divided by (homologous long RTL + homologous short RTL)
†all p-values were based on Student t-test except for 9p
*Wilcoxon ranks um test was used
Bold p-values were significant at <0.01 level

3. Discussion

This data shows that, after adjustment for known breast cancer risk factors, shorter telomere lengths on chromosome Xp and 15p were significantly associated with an increased risk of breast cancer in pre-menopausal women. These data support the hypothesis that women who have telomere length deficiency on certain chromosome arms are at increased risk of breast cancer. The present study is the first study that examined the association between all 92 individual telomeres in the human genome and risk of breast cancer. The results provided new evidence that short telomere lengths on chromosomes Xp and 15p were significantly associated with breast cancer risk in pre-menopausal women. The data also revealed that telomere lengths between non-homologous telomeres were not correlated and are likely independent genetic events that may carry information of clinical importance for cancer patients.

Deficiencies in telomere health is particularly relevant to carcinogenesis because hyper-proliferative cancerous cells could lead to progressive telomere shortening, ultimately generating uncapped telomeres that fuse with each other leading to genomic instability that promotes malignant transformation. However, it has not been established if it is the shortest telomeres or the mean telomere length that triggers the telomere dysfunction-associated responses. The discoveries and results described herein support the former mechanism. The discoveries and results described herein are also consistent with the report that individual dysfunctional telomeres are recognized as DNA damage and a cellular response is triggered (Artandi, 2005). Crossing telomerase knockout mice having short telomeres with those having long telomeres revealed that loss of telomere function occurs preferentially on the shortest telomere and that the shortest telomeres, rather than the average telomere length, elicit a cellular response (Hemann, 2001). The discoveries and results described herein are also consistent with reports that chromosome arms carrying the shortest telomeres were more often found in telomere fusions leading to chromosomal instability (Der-Sarkissian, 2004; Soler, 2005) (Capper R et al 2007, 21:2495). The discoveries and results described herein are also consistent with reports that, in humans, chromosome specific telomere lengths are highly variable between chromosomal arms (Gilson, 2007; Lansdorp, 1996; Graakjaer, 2003; Martens, 1998). The chromosome arm-specific telomere length polymorphism disclosed herein indicates that chromosome arms bearing the shortest telomeres may predispose to the chromosome alterations and therefore have an impact on the evolution of tumors. Regardless of this mechanism, the disclosed results and discoveries show that certain measures of individual telomere length provide an indication of cancer risk.

The comprehensive approaches used in this example allowed for examination of the associations between telomere length variations and breast cancer risk. One of the main efforts of telomere maintenance is to provide homogenous protection for all the telomeres and to minimize the opportunity for induction of dysfunctional telomeres. It was realized that high degree telomere length variations represent a deficiency in telomere maintenance and are linked to cancer susceptibility. To demonstrate this, the association between homologous telomere length difference (HTLD) and breast cancer risk were examined. The data indicated that greater HTLDs on chromosome 9p, 15p and 15q were significantly associated with breast cancer risk in pre-menopausal women. These data indicated that telomere length variations between homologous telomeres can represent different phenotypes of telomere deficiency that is linked to breast cancer susceptibility. This example introduces homologous telomere length difference as a new phenotype of deficiencies in telomere maintenance and identified greater HTLDs on chromosome 9p, 15p and 15q as new risk factors for breast cancer in premenopausal women.

This example also revealed that mean telomere length variation of 46 chromosome arms in lymphocytes is significantly higher in cases than in controls (p=1.50×10⁻⁷) in pre-menopausal women (Table 11). Examining individual telomere length variation in lymphocytes identified telomere length variation on 18p showing suggestive association with breast cancer risk in premenopausal women (Table 12). These data provided further evidence that greater telomere length heterogeneity can contribute to an increased breast cancer risk in premenopausal women.

Telomere capping presents a unique challenge to a proliferative cell. Because telomerase is nonessential and its activity is undetectable in most normal human somatic cells, an alternative mechanism that contributes to telomere maintenance may play the key role for telomere homeostasis in normal somatic cells. There is a growing body of evidence that homologous recombination (HR) proteins and other proteins involved in DNA repair have a complex role in normal telomere biology (Sarthy J 2009, 29:3390; Wu Y 2008, 129:602; Zeng S 2009, 11:616; Opresko P L 2004, 14:763; Poulet A 2009, 28:641; Verdum R E 2006, 127:709; Wang R C 2004, 119:355). During DNA replication, telomeres cycle through what appears to be recognition of chromosome ends as DNA damage during specific phases of the cell cycle (Verdun R E, 2005, 20:551; Verdun R E, 2006, 127:709). While the action of telomere binding proteins inhibit end-to-end fusions (Bae and Baumann 2007, 26:323), the temporary recruitment of HR proteins to the telomeres in S phase and early G2 phases is necessary for the HR-assisted capping during G2 to restructure the chromosome terminus into a t-loop, preventing the recognition of chromosome ends as DSBs in G1 (Verdun R E, 2005, 20:551; Verdun R E, 2006, 127:709). Therefore, well controlled moderate telomere HR is beneficial to capping-related roles. In the absence of proper regulation, telomere HR could result in several deleterious consequences, including telomere rapid deletions, recombinational telomere elongation or immortalization via the alternative lengthening of telomeres (ALT) pathway (De Boeck G 2009, 217:327). ALT mechanism may arise via a loss of function in the complex controls over telomere capping, leading to a telomere-specific increases in HR (Jiang W Q 2005, 25:2708; Potts P R 2007, 14:581; Zhong Z H 2007, 282:29314).

A substantial number of human malignant tumors utilize ALT, a telomerase-independent telomere length maintenance mechanism. These include bone and soft tissue sarcomas, glioblastomas, and carcinomas of the lung, kidney, breast and ovary (Bryan tm 1997, 3:1271; Mehle c 1996, 13:161). ALT-mediated telomere length maintenance is characterized by highly heterogeneous telomeric DNA (Bryan tm 1995, 14:4240; Henson J D 2002, 21:598; Cesare A J 2004, 24:9948). ALT telomere length dynamics are complex, with both rapid elongation and rapid deletion events superimposed on a background of constant telomere attrition (Murnane J P 1994, 13:4953). Telomere FISH showed that within any ALT cell, there are telomeres that ranged from <2 kb to >50 kb in length, and often several chromosome ends lacking any telomere signal (Henson J D 2002, 21:598). Given the important role of HR in normal telomere biology, it is possible that dysregulation of HR-assisted telomere maintenance can result in increased telomere variations between individual telomeres in somatic cells, resulting in an increased risk of cancer. The observation herein that greater length variations between homologous telomeres and among somatic cells are associated with an increased risk of breast cancer in pre-menopausal women is consistent with this mechanism.

The data in this example indicates that the associations between telomere deficiencies and breast cancer risk in pre-menopausal women only involve a handful of chromosome arms (Xp, 9p, 15p, 15q and 18p). The reason why these chromosomal arms are associated with this cancer risk may be related to telomere-mediated dysregulation of genes that reside on those chromosome arms and that are involved in breast carcinogenesis. For example, telomere lengths are shown to be the critical players in regulating epigenetic modification of regional chromatin and these telomere-related epigenetic changes could result in epigenetic dysregulation of oncogenes and/or tumor suppressor genes (Benetti, 2007; Garcia-Cao, 2004; Vera, 2008). Deficiency in 9p telomeres could potentially affect the stability of chromosome 9p, where the CDKN2A locus (also known as INK4a/ARF locus) locates at 9p21. CDKN2A locus encodes two proteins, p16^{INK4a} and p14^{ARF}, that regulate 2 critical cell cycle regulatory pathways: the p53 pathway and the retinoblastoma pathway (Harris, 2005; Sherr, 1996; Sherr, 1998). Inactivation of CDKN2A locus removes an important barrier to tumor progression and 9p21 is a frequent target of inactivation by deletion or aberrant DNA methylation in a wide variety of human cancers (Kim, 2006 1158/id), including breast cancer (Ellsworth, 2007; Hwang, 2004; Tao, 2009; Esteller, 2001; Gorgoulis, 1998). Despite its importance in tumor suppression and considerable research, the cause of CDKN2A inactivation by deletion or aberrant promoter methylation is still unknown.

The data in this example indicated that the association between chromosome arm specific telomere deficiencies and breast cancer risk is restricted to pre-menopausal women. In post-menopausal women, there is a suggestive association between greater telomere length variation in somatic cells on chromosomes 15p and 21p and decreased breast cancer risk. However, it should be noted that none of the associations in post-menopausal women were statistically significant after considering Bonferroni correction for multiple comparisons.

Given that this is a case-control study, there could have been a theoretical concern is that telomere length in lymphocytes is affected by case status (reverse causality). However, reverse causality is not a plausible explanation for the results. Data by previous studies and current studies indicated that the mean overall telomere length of blood leucocytes in breast cancer patients was not significantly shorter than in healthy women controls (Zheng, 2009; De, 2009; Shen, 2007; Barwell, 2007), suggesting there is no significant shortening of blood leucocyte telomere length associated with having breast cancer. Although previous studies (Schroder, 2001; Yoon, 2007) suggested that chemotherapy and/or radiotherapy can induce telomere shortening in leucocytes, all the blood samples in this example were drawn before any chemotherapy and radiotherapy treatments. Thus reverse causality is not a plausible explanation for the results. This example is limited by its moderate sample size and is not powered to detect the small to moderate associations (i.e. OR<2.0).

In summary, this example revealed that short telomere length on chromosome Xp and 15p, greater length differences between homologous telomeres on chromosome 9p, 15p and 15q, and greater telomere length variation in lymphocytes on chromosome 18p were significantly associated with breast cancer risk in premenopausal women. These data provided first evidence that telomere deficiency (poor telomere health) on certain chromosome arms are linked to breast cancer susceptibility. As described elsewhere herein, these new discoveries have clinical application in detecting and assessing cancer risk. Telomere-related parameters can be used as a panel of blood-based biomarkers for breast cancer risk assessment, given their strong associations with breast cancer risk. Better risk assessment would improve the efficiency of both population-based preventive programs, such as screening mammography, as well as individual-based preventive strategies such as chemoprevention by targeting women who are at the greatest risk for breast cancer.

H. References
(1) Parkin D M, Fernandez L M. Use of statistics to assess the global burden of breast cancer. Breast J 2006; 12 Suppl 1:S70-S80.
(2) Stewart S L, King J B, Thompson T D, Friedman C, Wingo P A. Cancer mortality surveillance-United States, 1990-2000. MMWR 2004; 53(SSO₃):1-108.
(3) Smigal C, Jemal A, Ward E et al. Trends in breast cancer by race and ethnicity: update 2006. CA Cancer J Clin 2006; 56(3):168-183.
(4) Familial breast cancer: collaborative reanalysis of individual data from 52 epidemiological studies including 58,209 women with breast cancer and 101,986 women without the disease. Lancet 2001; 358(9291):1389-1399.
(5) Baudis M. Genomic imbalances in 5918 malignant epithelial tumors: an explorative meta-analysis of chromosomal CGH data. BMC Cancer 2007; 7:226.
(6) Shih I M, Zhou W, Goodman S N, Lengauer C, Kinzler K W, Vogelstein B. Evidence that genetic instability occurs at an early stage of colorectal tumorigenesis. Cancer Res 2001; 61(3):818-822.
(7) Michor F, Iwasa Y, Vogelstein B, Lengauer C, Nowak M A. Can chromosomal instability initiate tumorigenesis? Semin Cancer Bio12005; 15(1):43-49.
(8) Hernando E, Nahle Z, Juan G et al. Rb inactivation promotes genomic instability by uncoupling cell cycle progression from mitotic control. Nature 2004; 430(7001):797-802.
(9) Fridlyand J, Snijders A M, Ylstra B et al. Breast tumor copy number aberration phenotypes and genomic instability. BMC Cancer 2006; 6:96.
(10) Gorgoulis V G, Koutroumbi E N, Kotsinas A et al. Alterations of p16-pRb pathway and chromosome locus 9p21-22 in sporadic invasive breast carcinomas. Mol Med 1998; 4(12):807-822.
(11) Miracca E C, Yamamoto L, Contado D, de Souza Silva T M, Kowalski L P, Nagai M A. Distinct chromosomal deleted regions defining different subsets of head and neck tumors. Diagn Mol Pathol 2000; 9(4):229-233.
(12) Mhawech-Fauceglia P, Cheney R T, Schwaller J. Genetic alterations in urothelial bladder carcinoma: an updated review. Cancer 2006; 106(6):1205-1216.
(13) Sato M, Takahashi K, Nagayama K et al. Identification of chromosome arm 9p as the most frequent target of homozygous deletions in lung cancer. Genes Chromosomes Cancer 2005; 44(4):405-414.
(14) Rakosy Z, Vizkeleti L, Ecsedi S et al. Characterization of 9p21 copy number alterations in human melanoma by fluorescence in situ hybridization. Cancer Genet Cytogenet 2008; 182(2):116-121.
(15) Ellsworth R E, Ellsworth D L, Love B et al. Correlation of levels and patterns of genomic instability with histological grading of D CIS Ann Surg Oncol 2007; 14(11):3070-3077.
(16) Hwang E S, DeVries S, Chew K L et al. Patterns of chromosomal alterations in breast ductal carcinoma in situ. Clin Cancer Res 2004; 10(15):5160-5167.
(17) An H X, Niederacher D, Picard F, van R C, Bender H G, Beckmann M W. Frequent allele loss on 9p21-22 defines a smallest common region in the vicinity of the CDKN2 gene in sporadic breast cancer. Genes Chromosomes Cancer 1996; 17(1):14-20.
(18) Xie D, Jauch A, Miller C W, Bartram C R, Koeffler H P. Discovery of over-expressed genes and genetic alterations in breast cancer cells using a combination of suppression subtractive hybridization, multiplex FISH and comparative genomic hybridization. Int J Oncol 2002; 21(3):499-507.
(19) Williamson M P, Elder P A, Shaw M E, Devlin J, Knowles M A. p16 (CDKN2) is a major deletion target at 9p21 in bladder cancer. Hum Mol Genet. 1995; 4(9):1569-1577.
(20) Cairns P, Polascik T J, Eby Y et al. Frequency of homozygous deletion at p16/CDKN2 in primary human tumours. Nat Genet. 1995; 11(2):210-212.
(21) Harris S L, Levine A J. The p53 pathway: positive and negative feedback loops. Oncogene 2005; 24(17):2899-2908.
(22) Gisselsson D, Jonson T, Petersen A et al. Telomere dysfunction triggers extensive DNA fragmentation and evolution of complex chromosome abnormalities in human malignant tumors. Proc Natl Acad Sci U SA 2001; 98(22):12683-12688.
(23) Zheng Y L, Loffredo C A, Alberg A J et al. Less efficient g2-m checkpoint is associated with an increased risk of lung cancer in African Americans. Cancer Res 2005; 65(20):9566-9573.
(24) Meeker A K, Hicks J L, Gabrielson E, Strauss W M, De Marzo A M, Argani P. Telomere shortening occurs in subsets of normal breast epithelium as well as in situ and invasive carcinoma. Am J Pathol 2004; 164(3):925-935.
(25) Meeker A K, Argani P. Telomere shortening occurs early during breast tumorigenesis: a cause of chromosome destabilization underlying malignant transformation? J Mammary Gland Biol Neoplasia 2004; 9(3):285-296.
(26) Poonepalli A, Banerjee B, Ramnarayanan K, Palanisamy N, Putti T C, Hande M P. Telomere-mediated genomic instability and the clinico-pathological parameters in breast cancer. Genes Chromosomes Cancer 2008; 47(12):1098-1109.
(27) Chin K, de Solorzano C O, Knowles D et al. In situ analyses of genome instability in breast cancer. Nat Genet. 2004; 36(9):984-988.
(28) Hemann M T, Strong M A, Hao L Y, Greider C W. The shortest telomere, not average telomere length, is critical for cell viability and chromosome stability. Cell 2001; 107(1):67-77.
(29) Lansdorp P M, Verwoerd N P, van de Rijke F M et al. Heterogeneity in telomere length of human chromosomes. Hum Mol Genet. 1996; 5(5):685-691.
(30) Graakjaer J, Bischoff C, Korsholm L et al. The pattern of chromosome-specific variations in telomere length in humans is determined by inherited, telomere-near factors and is maintained throughout life. Mech Ageing Dev 2003; 124(5):629-640.
(31) Martens U M, Zijlmans J M, Poon S S et al. Short telomeres on human chromosome 17p. Nat Genet. 1998; 18(1):76-80.
(32) Der-Sarkissian H, Bacchetti S, Cazes L, Londono-Vallejo J A. The shortest telomeres drive karyotype evolution in transformed cells. Oncogene 2004; 23(6):1221-1228.
(33) Shen J, Terry M B, Gurvich I, Liao Y, Senie R T, Santella R M. Short telomere length and breast cancer risk: a study in sister sets. Cancer Res 2007; 67(11):5538-5544.
(34) Svenson U, Nordfjall K, Stegmayr B et al. Breast cancer survival is associated with telomere length in peripheral blood cells. Cancer Res 2008; 68(10):3618-3623.
(35) Barwell J, Pangon L, Georgiou A et al. Is telomere length in peripheral blood lymphocytes correlated with cancer susceptibility or radiosensitivity? Br J Cancer 2007; 97(12):1696-1700.
(36) Schroder C ID, Wisman G B, de J S et al. Telomere length in breast cancer patients before and after chemotherapy with or without stem cell transplantation. Br J Cancer 2001; 84(10):1348-1353.
(37) Yoon S Y, Sung H J, Park K H et al. Telomere length shortening of peripheral blood mononuclear cells in solid-cancer patients undergoing standard-dose chemotherapy might be correlated with good treatment response and neutropenia severity. Acta Haematol 2007; 118(1):30-37.
(38) Hackett, J. A., Feldser, D. M., and Greider, C. W. (2001) Telomere dysfunction increases mutation rate and genomic instability. Cell, 106, 275-286.
(39) Stewenius, Y., Gorunova, L., Jonson, T., Larsson, N., Hoglund, M., Mandahl, N., Mertens, F., Mitelman, F., and Gisselsson, D. (2005) Structural and numerical chromosome changes in colon cancer develop through telomere-mediated anaphase bridges, not through mitotic multipolarity. Proc. Natl. Acad Sci U.S A, 102, 5541-5546.
(40) Lo, A. W., Sabatier, L., Fouladi, B., Pottier, G., Ricoul, M., and Murnane, J. P. (2002) DNA amplification by breakage/fusion/bridge cycles initiated by spontaneous telomere loss in a human cancer cell line. Neoplasia, 4, 531-538.
(41) McEachern, M. J., Krauskopf, A., and Blackburn, E. H. (2000) Telomeres and their control. Annu. Rev Genet, 34, 331-358.
(42) Maser, R. S, and DePinho, R. A. (2002) Connecting chromosomes, crisis, and cancer. Science, 297, 565-569.
(43) Gilson, E. and Londono-Vallejo, A. (2007) Telomere length profiles in humans: all ends are not equal. Cell Cycle, 6, 2486-2494.
(44) Zheng, Y-L., Ambrosone, C. B., Byrne, C., Davis, W., Nesline, M., and McCann, S. E. (2009) Telomere length from blood cells and breast cancer risk: investigations in two case-control studies. Breast Cancer Res Treat, in press.
(45) De, V., I, Prescott, J., Wong, J. Y., Kraft, P., Hankinson, S. E., and Hunter, D. J. (2009) A prospective study of relative telomere length and postmenopausal breast cancer risk. Cancer Epidemiol. Biomarkers Prev., 18, 1152-1156.
(46) Graakjaer, J., Der-Sarkissian, H., Schmitz, A., Bayer, J., Thomas, G., Kolvraa, S., and Londono-Vallejo, J. A. (2006) Allele-specific relative telomere lengths are inherited. Hum. Genet., 119, 344-350.

1-74. (canceled)

75. A method of assaying a subject comprising,
(a) Obtaining a sample from the subject;
(b) Measuring a telomere ratio from a cell from the sample; and
(c) Identifying a subject having a telomere ratio less than a reference telomere ratio.

76. The method of claim 75, wherein a subject having a telomere ratio less than a reference telomere ratio has an increased likelihood of having cancer.

77. The method of claim 75, wherein a subject having a telomere ratio less than a reference telomere ratio has an increased likelihood of contracting cancer.

78. The method of claim 77, wherein the likelihood indicates at least a 3.0, 3.9, or 6.6, fold increase relative to all women.

79. The method of claim 77, wherein the likelihood indicates at least a 2.1, 2.9, or 6.2, fold increase relative to all premenopausal women.

80. The method of claim 77, wherein the likelihood indicates at least a 4.3, 5.1, or 7.5, fold increase relative to all postmenopausal women.

81. The method of claim 77, wherein the cancer is breast cancer.

82. The method of claim 77, wherein the reference telomere ratio is 0.00494, 0.00583, or 0.00680.

83. The method of claim 75, wherein measuring the telomere ratio comprises substituting an indirect measurement of telomere length represented in the telomere ratio.

84. The method of claim 75, wherein measuring the telomere ratio comprises substituting an indirect measurement of telomere length for all telomeres represented in the telomere ratio.

85. The method of claim 84, wherein the indirect measurement comprises measuring a telomere marker.

86. The method of claim 85, wherein the telomere marker comprises a telomere hybridization probe.

87. The method of claim 86, wherein the hybridization probe comprises a fluorescent probe.

88. The method of claim 84, wherein the indirect measurement comprises the fluorescent signal of a fluorescent in situ hybridization assay.

89. The method of claim 75, wherein measuring the telomere ratio comprises measuring the length of at least one telomere represented in the telomere ratio.

90. The method of claim 75, wherein measuring the telomere ratio comprises measuring the length of all telomeres represented in the telomere ratio.

91. The method of claim 75 further comprising the step of measuring the length of all of the telomeres in the cell of the subject, producing a total telomere length.

92. The method of claim 75 further comprising the step of creating a telomere ratio.

93. A method of treating a patient comprising, analyzing the results of the method of claim 75, and based on the risk performing a treatment to reduce the likelihood of the subject getting cancer.

94. A method of treating a patient comprising, analyzing the results of the method of claim 75, and based on the risk performing a treatment to treat cancer in the subject.

95. A method of assaying the risk of cancer, in a subject, based on telomere length comprising:
(a) Taking a blood sample from the subject;
(b) Set up lymphocyte culture using fresh whole blood;
(c) Harvesting the chromosome preparation using standard cytogenetic method;
(d) Performing quantitative telomere fluorescent in situ hybridization (FISH);
(e) Analyzing FISH images of chromosome spreads to quantify telomere length (telomere signal intensity);
(f) Defining the relative telomere length of a chromosome as the intensity of telomere signal of the chromosome divided by the intensity of total telomere signals of the cell; and
(g) Determining the likelihood of getting cancer for an individual using statistic prediction model.
